SEQUENCE LISTING (56 sequences)

SEQ ID NO: 1 - DNA, 2221 bp, Homo sapiens
tcctacaagcagccggcggcgccgccgagtgaggggacgcggcgcggtggggcggcgcggcccgaggaggcggcggaggaggggccgcccgcggcccccggctcactccggcactccgggccgctcggcccccatgcctgcccgaccgcgctgccggagccccaggtgaccagcgccatgtccagccaggtggtgggcattgagcctctctacatcaaggcagagccggccagccctgacagtccaaagggttcctcggagacagagaccgagcctcctgtggccctggcccctggtccagctcccactcgctgcctcccaggccacaaggaagaggaggatggggagggggctgggcctggcgagcagggcggtgggaagctggtgctcagctccctgcccaagcgcctctgcctggtctgtggggacgtggcctccggctaccactatggtgtggcatcctgtgaggcctgcaaagccttcttcaagaggaccatccaggggagcatcgagtacagctgtccggcctccaacgagtgtgagatcaccaagcggagacgcaaggcctgccaggcctgccgcttcaccaagtgcctgcgggtgggcatgctcaaggagggagtgcgcctggaccgcgtccggggtgggcggcagaagtacaagcggcggccggaggtggacccactgcccttcccgggccccttccctgctgggcccctggcagtcgctggaggcccccggaagacagcagccccagtgaatgcactggtgtctcatctgctggtggttgagcctgagaagctctatgccatgcctgaccccgcaggccctgatgggcacctcccagccgtggctaccctctgtgacctctttgaccgagagattgtggtcaccatcagctgggccaagagcatcccaggcttctcatcgctgtcgctgtctgaccagatgtcagtactgcagagcgtgtggatggaggtgctggtgctgggtgtggcccagcgctcactgccactgcaggatgagctggccttcgctgaggacttagtcctggatgaagagggggcacgggcagctggcctgggggaactgggggctgccctgctgcaactagtgcggcggctgcaggccctgcggctggagcgagaggagtatgttctactaaaggccttggcccttgccaattcagactctgtgcacatcgaagatgccgaggctgtggagcagctgcgagaagctctgcacgaggccctgctggagtatgaagccggccgggctggccccggagggggtgctgagcggcggcgggcgggcaggctgctgctcacgctaccgctcctccgccagacagcgggcaaagtgctggcccatttctatggggtgaagctggagggcaaggtgcccatgcacaagctgttcttggagatgctcgaggccatgatggactgaggcaaggggtgggactggtgggggttctggcaggacctgcctagcatggggtcagccccaagggctggggcggagctggggtctgggcagtgccacagcctgctggcagggccagggcaatgccatcagcccctgggaacaggccccacgccctctcctccccctcctagggggtgtcagaagctgggaacgtgtgtccaggctctgggcacagtgctgccccttgcaagccataacgtgcccccagagtgtagggggccttgcggaagccatagggggctgcacgggatgcgtgggaggcagaaacctatctcagggagggaaggggatggaggccagagtctcccagtgggtgatgcttttgctgctgcttaatcctaccccctcttcaaagcagagtgggacttggagagcaaaggcccatgcccccttcgctcctcctctcatcatttgcattgggcattagtgtccccccttgaagcaataactccaagcagactccagcccctggacccctggggtggccagggcttccccatcagctcccaacgagcctcctcagggggtaggagagcactgcctctatgccctgcagagcaataacactatatttatttttgggtttggccagggaggcgcagggacatggggcaagccagggcccagagcccttggctgtacagagactctattttaatgtatatttgctgcaaagagaaaccgcttttggttttaaacctttaatgagaaaaaaatatataataccgagctc

SEQ ID NO: 2 - DNA, 2354 bp, Homo sapiens
tttttttcccctgctctcccaggggccagacaccaccgccccacccctcacgccccacctccctgggggatcctttccgccccagccctgaaagcgttaaccctggagctttctgcacaccccccgaccgctcccgcccaagcttcctaaaaaagaaaggtgcaaagtttggtccaggatagaaaaatgactgatcaaaggcaggcgatacttcctgttgccgggacgctatatataacgtgatgagcgcacgggctgcggagacgcaccggagcgctcgcccagccgccgcctccaagcccctgaggtttccggggaccacaatgaacaacttgctgtgctgcgcgctcgtgtttctggacatctccattaagtggaccacccaggaaacgtttcctccaaagtaccttcattatgacgaagaaacctctcatcagctgttgtgtgacaaatgtcctcctggtacctacctaaaacaacactgtacagcaaagtggaagaccgtgtgcgccccttgccctgaccactactacacagacagctggcacaccagtgacgagtgtctatactgcagccccgtgtgcaaggagctgcagtacgtcaagcaggagtgcaatcgcacccacaaccgcgtgtgcgaatgcaaggaagggcgctaccttgagatagagttctgcttgaaacataggagctgccctcctggatttggagtggtgcaagctggaaccccagagcgaaatacagtttgcaaaagatgtccagatgggttcttctcaaatgagacgtcatctaaagcaccctgtagaaaacacacaaattgcagtgtctttggtctcctgctaactcagaaaggaaatgcaacacacgacaacatatgttccggaaacagtgaatcaactcaaaaatgtggaatagatgttaccctgtgtgaggaggcattcttcaggtttgctgttcctacaaagtttacgcctaactggcttagtgtcttggtagacaatttgcctggcaccaaagtaaacgcagagagtgtagagaggataaaacggcaacacagctcacaagaacagactttccagctgctgaagttatggaaacatcaaaacaaagaccaagatatagtcaagaagatcatccaagatattgacctctgtgaaaacagcgtgcagcggcacattggacatgctaacctcaccttcgagcagcttcgtagcttgatggaaagcttaccgggaaagaaagtgggagcagaagacattgaaaaaacaataaaggcatgcaaacccagtgaccagatcctgaagctgctcagtttgtggcgaataaaaaatggcgaccaagacaccttgaagggcctaatgcacgcactaaagcactcaaagacgtaccactttcccaaaactgtcactcagagtctaaagaagaccatcaggttccttcacagcttcacaatgtacaaattgtatcagaagttatttttagaaatgataggtaaccaggtccaatcagtaaaaataagctgcttataactggaaatggccattgagctgtttcctcacaattggcgagatcccatggatgagtaaactgtttctcaggcacttgaggctttcagtgatatctttctcattaccagtgactaattttgccacagggtactaaaagaaactatgatgtggagaaaggactaacatctcctccaataaaccccaaatggttaatccaactgtcagatctggatcgttatctactgactatattttcccttattactgcttgcagtaattcaactggaaattaaaaaaaaaaaactagactccattgtgccttactaaatatgggaatgtctaacttaaatagctttgagatttcagctatgctagaggcttttattagaaagccatatttttttctgtaaaagttactaatatatctgtaacactattacagtattgctatttatattcattcagatataagatttgtacatattatcatcctataaagaaacggtatgacttaattttagaaagaaaattatattctgtttattatgacaaatgaaagagaaaatatatatttttaatggaaagtttgtagcatttttctaataggtactgccatatttttctgtgtggagtatttttataattttatctgtataagctgtaatatcattttatagaaaatgcattatttagtcaattgtttaatgttggaaaacatatgaaatataaattatctgaatattagatgctctgagaaattgaatgtaccttatttaaaagattttatggttttataactatataaatgacattattaaagttttcaaattattttttaaaaaaaaa

SEQ ID NO: 3 - DNA, 3677 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 4 - DNA, 27 bp, Artificial
  Synthetic oligonucleotide primer
gggaagcttagcgccatgtccagccag

SEQ ID NO: 5 - DNA, 28 bp, Artificial
  Synthetic oligonucleotide primer
gggggatccccaccccttgcctcagtcc

SEQ ID NO: 6 - DNA, 27 bp, Artificial
  Synthetic oligonucleotide primer
gggggatcctcatgtctggcggaggag

SEQ ID NO: 7 - DNA, 20 bp, Artificial
  Synthetic oligonucleotide primer
caaggagctggaagtgctgc

SEQ ID NO: 8 - DNA, 20 bp, Artificial
  Synthetic oligonucleotide primer
cagctctttccacgatggct

SEQ ID NO: 9 - DNA, 21 bp, Artificial
  Synthetic oligonucleotide primer
accgagagattgtggtcacca

SEQ ID NO: 10 - DNA, 22 bp, Artificial
  Synthetic oligonucleotide primer
catccacacgctctgcagtact

SEQ ID NO: 11 - DNA, 19 bp, Artificial
  Synthetic oligonucleotide primer
tggtgtgcacaggagcaag

SEQ ID NO: 12 - DNA, 20 bp, Artificial
  Synthetic oligonucleotide primer
ttcacatcacagctccccac

SEQ ID NO: 13 - DNA, 20 bp, Artificial
  Synthetic oligonucleotide primer
cacgacaacatatgttccgg

SEQ ID NO: 14 - DNA, 22 bp, Artificial
  Synthetic oligonucleotide primer
tgtccaatgtgccgctgcacgc

SEQ ID NO: 15 - DNA, 20 bp, Artificial
  Synthetic oligonucleotide primer
cgccgaccaaggaaaactca

SEQ ID NO: 16 - DNA, 20 bp, Artificial
  Synthetic oligonucleotide primer
aacggggatggccttgtatg

SEQ ID NO: 17 - DNA, 18 bp, Artificial
  Synthetic oligonucleotide primer
aggaggagggcagaatca

SEQ ID NO: 18 - DNA, 23 bp, Artificial
  Synthetic oligonucleotide primer
tctatctttctttggtctgcatt

SEQ ID NO: 19 - DNA, 22 bp, Artificial
  Synthetic oligonucleotide primer
ccaggcccaggtattggagggg

SEQ ID NO: 20 - DNA, 19 bp, Artificial
  Synthetic oligonucleotide primer
ggccgagttcatgagccgc

SEQ ID NO: 21 - DNA, 22 bp, Artificial
  Synthetic oligonucleotide primer
ggagtggacgaggcaagagttt

SEQ ID NO: 22 - DNA, 22 bp, Artificial
  Synthetic oligonucleotide primer
agcttctgtctgtgccttctgg

SEQ ID NO: 23 - DNA, 20 bp, Artificial
  Synthetic oligonucleotide primer
tagcaccttggatgggtatt

SEQ ID NO: 24 - DNA, 20 bp, Artificial
  Synthetic oligonucleotide primer
atcctgaggcacagtctgat

SEQ ID NO: 25 - DNA, 20 bp, Artificial
  Synthetic oligonucleotide primer
cttagaggtgactggcaaac

SEQ ID NO: 26 - DNA, 20 bp, Artificial
  Synthetic oligonucleotide primer
gcccatcaaatgggtagaag

SEQ ID NO: 27 - DNA, 22 bp, Artificial
  Synthetic oligonucleotide primer
gagccgcctccggagagagacg

SEQ ID NO: 28 - DNA, 20 bp, Artificial
  Synthetic oligonucleotide primer
tagggtctgggtgttcgatg

SEQ ID NO: 29 - DNA, 18 bp, Artificial
  Synthetic oligonucleotide primer
gggcagatgtctgcacag

SEQ ID NO: 30 - DNA, 21 bp, Artificial
  Synthetic oligonucleotide primer
cttgaagttcatcacctgccc

SEQ ID NO: 31 - DNA, 20 bp, Artificial
  Synthetic oligonucleotide primer
cctggctccttgggacccgt

SEQ ID NO: 32 - DNA, 20 bp, Artificial
  Synthetic oligonucleotide primer
atttgctgcacgctgccgtc

SEQ ID NO: 33 - DNA, 20 bp, Artificial
  Synthetic oligonucleotide primer
gacctcagctacatcgtgcg

SEQ ID NO: 34 - DNA, 20 bp, Artificial
  Synthetic oligonucleotide primer
cgtgctctggctcagatgcc

SEQ ID NO: 35 - DNA, 20 bp, Artificial
  Synthetic oligonucleotide primer
ggcaggtgctacagcgctcc

SEQ ID NO: 36 - DNA, 20 bp, Artificial
  Synthetic oligonucleotide primer
tccctgtccaccgcctgagc

SEQ ID NO: 37 - DNA, 20 bp, Artificial
  Synthetic oligonucleotide primer
gagttgggaggaggcaggcg

SEQ ID NO: 38 - DNA, 19 bp, Artificial
  Synthetic oligonucleotide primer
ggactgcaggcttcctgtg

SEQ ID NO: 39 - DNA, 19 bp, Artificial
  Synthetic oligonucleotide primer
ggctaactctgaggacacg

SEQ ID NO: 40 - DNA, 19 bp, Artificial
  Synthetic oligonucleotide primer
gatgtatctgataaacaag

SEQ ID NO: 41 - DNA, 23 bp, Artificial
  Synthetic oligonucleotide primer
ccaccgagacaccatgagagccc

SEQ ID NO: 42 - DNA, 19 bp, Artificial
  Synthetic oligonucleotide primer
ggggactggggctcccagc

SEQ ID NO: 43 - DNA, 20 bp, Artificial
  Synthetic oligonucleotide primer
caaggagctggaggtgctgc

SEQ ID NO: 44 - DNA, 19 bp, Artificial
  Synthetic oligonucleotide primer
ctgctctttctacaatggc

SEQ ID NO: 45 - DNA, 20 bp, Artificial
  Synthetic oligonucleotide primer
ggtgatagcttggcttatgg

SEQ ID NO: 46 - DNA, 19 bp, Artificial
  Synthetic oligonucleotide primer
ggcatgctcagaagctggg

SEQ ID NO: 47 - DNA, 20 bp, Artificial
  Synthetic oligonucleotide primer
cccgaatccttaagggccag

SEQ ID NO: 48 - DNA, 19 bp, Artificial
  Synthetic oligonucleotide primer
tatgcgatgtccttgcagc

SEQ ID NO: 49 - DNA, 22 bp, Artificial
  Synthetic oligonucleotide primer
tgcctacttttatcctcctctg

SEQ ID NO: 50 - DNA, 20 bp, Artificial
  Synthetic oligonucleotide primer
acccgagagtgtggaaagtg

SEQ ID NO: 51 - DNA, 20 bp, Artificial
  Synthetic oligonucleotide primer
tgacaaagccttcatgtcca

SEQ ID NO: 52 - DNA, 20 bp, Artificial
  Synthetic oligonucleotide primer
gagaggacagggaggatcaa

SEQ ID NO: 53 - DNA, 21 bp, Artificial
  Synthetic oligonucleotide primer
gtggtctgcaggatcgctctg

SEQ ID NO: 54 - DNA, 19 bp, Artificial
  Synthetic oligonucleotide primer
cgctgggccacatccaacc

SEQ ID NO: 55 - DNA, 19 bp, Artificial
  Synthetic oligonucleotide primer
tgtgtgacaaatgtgctcc

SEQ ID NO: 56 - DNA, 20 bp, Artificial
  Synthetic oligonucleotide primer
gtctcacctgagaagaaccc

(19) United States
(12) Patent Application Publication
(10) Pub. No.: US 2013/0316359 A1
(43) Pub. Date: Nov. 28, 2013

(54) METHOD FOR DETERMINING CANCER PROGNOSIS

(71) Applicants: Edith Bonnelye, Lyon (FR); Anais Fradet, Lyon (FR); Philippe Clezardin, Lyon (FR)

(72) Inventors: Edith Bonnelye, Lyon (FR); Anais Fradet, Lyon (FR); Philippe Clezardin, Lyon (FR)

(73) Assignees: INSTITUT NATIONAL DE LA SANTE ET DE LA RECHERCHE MEDICALE, Paris (FR); UNIVERSITE CLAUDE BERNARD LYON 1, Lyon Cedex 07 (FR)

(21) Appl. No.: 13/982,843

(22) PCT Filed: Jan. 27, 2012
(86) PCT No.: PCT/EP2012/051323

§ 371 Date: Jul. 31, 2013

(30) Foreign Application Priority Data
Jan. 31, 2011 (EP) 11305094.2

(51) Int. Cl. C12Q 1/68; G01N 33/68

(52) U.S. Cl. CPC C12Q 1/6886; G01N 33/6893
USPC 435/6.12; 435/7.1; 435/7.92

(57) ABSTRACT

The present invention concerns an in vitro method for determining cancer prognosis for a patient suffering from early-stage or low-grade cancer, said method comprising measuring the expression level of ERRα in a biological sample comprising cancer cells. The invention further pertains to an in vitro method for determining bone metastases prognosis for a patient suffering from bone metastases comprising measuring the expression level of ERRα. Finally, the invention pertains to in vitro methods for selecting a patient suffering from cancer, and/or from cancer-derived metastasis, suitable to be treated with a preventive/aggressive therapy.

The present invention concerns an in vitro method for determining cancer prognosis for a patient suffering from early-stage or low-grade cancer, in particular from breast cancer, said method comprising measuring the expression level of ERRα in a biological sample comprising cancer cells. The invention further pertains to an in vitro method for determining bone metastases prognosis for a patient suffering from cancer-derived bone metastases comprising measuring the expression level of ERRα. Finally, the invention pertains to in vitro methods for selecting a patient suffering from cancer, and/or from cancer-derived metastasis, suitable to be treated with a preventive or aggressive therapy.

### BACKGROUND OF THE INVENTION

Most breast cancers are epithelial tumors. Cancers are divided into carcinoma in situ and invasive cancer. Breast cancer invades locally and spreads initially through the regional lymph nodes, bloodstream, or both. Metastatic breast cancer may affect almost any organ in the body, most commonly, lungs, liver, bone, brain, and skin. Metastatic breast cancer frequently appears years or decades after initial diagnosis and treatment.

Diagnosis of breast cancer is mainly performed by screening by mammography, breast examination, or sometimes magnetic resonance imaging. Analysis of a biopsy, including analysis for estrogen and progesterone receptors and for HER2 protein, may also allow diagnosis of breast cancer.

Long-term prognosis depends on the tumor. Currently, nodal status (including number and location of nodes) correlates with disease-free and overall survival better than any other prognostic factor.

Patients with estrogen receptors (ER+) tumors have a somewhat better prognosis and are more likely to benefit from hormone therapy. Patients with progesterone receptors on a tumor may also have a better prognosis. Patients with both estrogen and progesterone receptors on a tumor may have a better prognosis than those who have only one of these receptors, but this is not clear.

When the HER2 gene (HER2/neu or ErbB2) is amplified, HER2 is overexpressed, increasing cell growth and reproduction and often resulting in more aggressive tumor cells. Overexpression of HER2 is an independent risk factor for a poor prognosis; it also may be associated with high histologic grade, ER-tumors, greater proliferation, and larger tumor size, all of them being poor prognostic factors.

For any given stage, patients with mutations in the BRCA1 gene appear to have a worse prognosis than those with sporadic tumors, perhaps because they have a higher proportion of high-grade, hormone receptor-negative cancers. Patients with mutations in the BRCA2 gene probably have the same prognosis as those without mutations if the tumors have similar characteristics. With mutations in either gene, risk of a 2nd cancer in remaining breast tissue is increased (to perhaps as much as 40%)

For most patients, primary treatment is surgery, often with radiation therapy. Chemotherapy, hormone therapy, or both, may also be used, depending on tumor and patient characteristics.

Bone metastases are a frequent complication of cancer, occurring in up to 70 percent of patients with advanced breast cancer. Bone metastases are not a direct cause of death but are associated with significant morbidity such as bone pain, impaired mobility, hypercalcaemia, pathological fracture and spinal cord compression. For cancer cells to grow in bone, malignant cells recruit and activate osteoclasts (bone resorbing cells) to resorb the bone matrix. For example, osteolytic breast cancer metastases are characterized by an increase in osteoclast number and activity at the bone metastatic site, where excessive bone destruction provides a permissive microenvironment for breast cancer cells to proliferate and expand. Unfortunately, current treatments for bone metastases that rely on anti-resorptive agents are only palliative, raising the need for a better understanding of the molecular mechanisms involved in this pathology so as to design potential alternative therapies.

Nuclear steroid receptors are transcription factors that comprise both ligand-dependent molecules such as estrogen receptors (ERs) and a large number of so-called orphan receptors, for which no ligands have yet been determined. Three orphan receptors, estrogen receptor-related receptor α (ERRα), ERRβ and ERRγ, (NR3B1 NR3B2 and, NR3B3 respectively, according to the Nuclear Receptors Nomenclature Committee, 1999), share structural similarities with ERα and ERβ (NR3A1 and NR3A2 respectively), but they do not bind estrogen. Sequence alignment of ERRα and the ERs reveals a high similarity (68%) in the 66 amino acids of the DNA binding domain, but only a moderate similarity (36%) in the ligand-binding domain, which may explain the fact that ERRα recognizes the same DNA binding elements as ERs but does not bind estrogen. Although ERRα activity is decreased by the synthetic molecule XCT790, no natural ligand has yet been found.

ERRα is known to regulate fatty acid oxidation and the adaptative bioenergetic response. It is widely expressed in normal tissues and several recent RNA expression studies show its presence in a range of cancerous cells including breast, prostate, endometrial, colorectal and ovarian tumour tissues. ERRα is markedly increased in tumour versus normal tissues and, in advanced cancers, ERRα-positive cancers (breast, ovarian colorectal) are associated with more aggressive disease and poorer patient prognosis that included an increased risk of recurrence (Suzuki et al, 2004, Estrogen-related receptor alpha in human breast carcinoma as a potent prognostic factor; cancer Res, 64 (13): 4670-6; Ariazi, et al, 2002, Estrogen-related receptor alpha and estrogen-related receptor gamma associate with unfavorable and favorable biomarkers, respectively, in human breast cancer; Cancer Res; 62(22):6510-18). On the other hand, ERα and ERβ are significantly lower in tumours versus normal tissue and are associated with better prognosis, as the expression of both receptors is lower in aggressive stages of the disease (Ariazi, et al, 2002, Estrogen-related receptor alpha and estrogen-related receptor gamma associate with unfavorable and favorable biomarkers, respectively, in human breast cancer; Cancer Res; 62(22):6510-18). ERRα is also highly expressed in skeletal (bone and cartilage) tissues. Its expression in osteoprogenitors, proliferating and differentiating osteoblasts in primary rat calvaria cell cultures correlates with its detection in bone in vivo. Moreover, ERRα has been reported to regulate osteoblast and osteoclast development and bone formation in vitro and in vivo. Consistent with these observations, osteopontin (OPN) has been reported to be a direct target gene of ERRα in osteoblastic cell lines (Bonnelye et al, 1997, The ERR-1 orphan receptor is a transcriptional activator expressed during bone development, Mol Endocrinol, 11(7): 905-916).

Today, prognosis of well-established cancer based on nodal status and estrogen receptor observation is well-known by the skilled in the art. However, prognosis is difficult to establish when the cancer is an early-stage or low-grade cancer. Therefore, there is a need for a method for prognosing early-stage or low-grade cancers, especially in the case of pN0 and ER+ cancers.

### DESCRIPTION OF THE INVENTION

The inventors have reported the first evidence of a statistical association between ERRα expression and VEGF expression and between ERRα and OPG in a cohort of breast cancer patients (N=251). Moreover, high ERRα expression also correlated with a higher risk of recurrence at a very early stage of the disease when patients belong to groups of good prognostic (ER+ and pN0). The median value of ERRα in the pN0 subset was also significantly associated with ERs and VEGF. Moreover, they have found an association between ERRα and VEGF, and ERRα and OPG, and the increased size and vascularity of primary tumors in mice inoculated with ERRα-overexpressing breast cancer cells. Interestingly, mice inoculated with ERRα-overexpressing breast cancer cells revealed a decrease in the osteolytic lesions development probably due to the association between ERRα and OPG. These results support that ERRα is a pro-angiogenic factor and an unfavorable prognostic factor in primary breast cancer and their metastases, with the exception of bone metastases, wherein ERRα is a good prognostic factor.

Indeed, the inventors have also reported the first evidence that ERRα is involved in development of bone metastases. ERRα over-expression decreased breast cancer cell-induced osteolytic lesion size, inhibited osteoclats (OCs) formation and altered expression of a variety of osteoblasts (OBs) markers, including the main OCs inhibitor OPG (also know to be a pro-angiogenic factor) in breast cancer cells. The low impact of VEGF and OPG on angiogenesis in bone, together with the ability of ERRα to up-regulate OPG and decrease osteoclastogenesis and overall bone remodeling, support a protective and favorable role for ERRα in osteolytic lesions and bone metastases development.

In summary, the inventors of the present patent application have surprisingly found that ERRα is a marker of poor prognosis for early-stage and low-grade cancer. That is to say ERRα can be seen as a prevention marker. When it is expressed at high levels, the patients should be placed under very tight observation by their oncologist, although the patients suffer from an early-stage and/or a low-grade cancer.

Prognosis of Patients Suffering from Early-Stage and/or Low-Grade Cancer

Therefore, the present invention provides an in vitro method for determining the prognosis of a patient suffering from early-stage and/or low-grade cancer, said method comprising:

a) providing or obtaining a biological sample comprising cancer cells from said patient;

b) measuring the expression level of Estrogen-Related Receptor α (ERRα) in said biological sample; and

c) optionally deducing from the result of step b) the prognosis of said patient.

As used throughout the present specification, the term “ERRα” refers to the human estrogen receptor-related receptor α protein. This term is meant to encompass any naturally occurring isoform of the ERRα protein, including the protein having an amino acid sequence of SEQ ID NO: 1, allelic variants thereof and splice variants thereof. In a preferred embodiment, measuring the expression level of ERRα comprises or consists of measuring the expression level of an ERRα protein of SEQ ID NO: 1.

As used throughout the present specification, the term “cancer” refers to any type of malignant (i.e. non benign) tumor, such as e.g. breast cancer. Further, the tumor may correspond to a solid malignant tumor, which includes e.g. carcinomas, adenocarcinomas, sarcomas, melanomas, mesotheliomas, blastomas, or to a blood cancer such as leukaemias, lymphomas and myelomas. The cancer may for example correspond to breast cancer, endometrial cancer, ovarian cancer, prostate cancer, lung cancer, colorectal cancer, or glioma.

According to the invention, the cancer can be a low-grade cancer. Tumor “grade” is a system used to classify cancer cells in terms of how abnormal they appear and how quickly the tumor is likely to grow and spread. Many factors are considered when determining tumor grade, including the structure and growth pattern of the cells. More specifically, cancer cells are “low grade” if they appear similar to normal cells, and “high grade” if they appear poorly differentiated. For example, a G1 cancer would be classified as a low-grade cancer, whereas a G4 cancer would be classified as a high-grade cancer.

Additionally or alternatively, the cancer can be an early-stage cancer. Cancer “stage” refers to the extent or severity of the cancer, based on factors such as the location of the primary tumor, tumor size, number of tumors, and lymph node involvement (spread of cancer into lymph nodes). For instance, an “early-stage” cancer is a single tumor, of small size, with a low degree of spread to regional lymph nodes. The stage of a cancer may for instance be determined using the TNM classification. In this classification, T(a,is,(0),1-4) indicates the size or direct extent of the primary tumor, N(0-3) indicates the degree of spread to regional lymph nodes, and M(0/1) indicates the presence of metastasis. For example, a T1/N0/M0 cancer would be classified as a early-stage cancer, whereas a T4/N3/M1 cancer would be classified as a late-stage cancer.

Until now, patients suffering from early-stage and/or low-grade cancer were believed to have a good prognosis. For example, nodal status (including number and location of nodes) correlates with disease-free and overall survival better than any other prognostic factor.

The inventors have surprisingly found that ERRα is a cancer marker of poor prognosis in a cohort comprising pN0 patients, pN<3 lymph-node positive patients and ER+ patients. That is to say, ERRα is an early marker allowing defining patients with a bad prognosis within a group of patients which were, until now, considered to have a good prognosis.

Therefore, in a specific embodiment, the patient suffering from early-stage and/or low-grade cancer and for which prognosis is sought is either a pN0 patient, or a pN<3 lymph-node positive patient.

In another specific embodiment, the early-stage and/or low-grade cancer for which prognosis is sought is a hormone-dependent cancer such as e.g. prostate cancer or breast cancer. In a preferred embodiment, the cancer is a breast cancer, and the patient suffering from cancer is an Estrogen Receptor-positive (ER+) patient. ER+ patients suffer from hormone-dependent breast cancer, i.e. from a cancer that is clinically defined as being hormone responsive.

Estrogen receptors are protein molecules that bind to the estrogen hormone. A breast cancer patient having estrogen receptors present on many of the cancer cells is considered as a “estrogen-receptor-positive” (ER+) patient. ER-positive cancers rely on a source of estrogen to encourage proliferation (increase the number) of cancer cells. Because of their dependency on estrogen, most ER-positive cancers respond well to anti-estrogen therapies, such as for instance Tamoxifen. The anti-estrogen therapies work by blocking the cancer cells' estrogen receptors, effectively cutting off their nourishment. Therefore, patients with ER+ tumors have a somewhat better prognosis and are more likely to benefit from hormone therapy. Patients with progesterone receptors on a tumor may also have a better prognosis. Patients with both estrogen and progesterone receptors on a tumor may have a better prognosis than those who have only one of these receptors. However, the inventors have found that such patients, currently believed to have a good prognosis, have a poor prognosis if ERRα is expressed.

The results presented herein show that ERRα is an unfavorable prognostic biomarker in breast cancer, including at a very early stage of the disease when patients belong to groups of good prognostic (e.g. ER+ and pN0 patients). A statistical association between ERRα expression and histological type and node status in a cohort of breast cancer patients (N=251) is also reported. Therefore, the measurement of a high expression level of ERRα is indicative of a poor prognosis.

In a specific embodiment of the in vitro method for determining the prognosis of a patient suffering from early-stage and/or low-grade cancer, an expression level of ERRαhigher than a predetermined threshold is indicative of a poor prognosis.

As used throughout the present specification, the term “poor prognosis” refers to a patient that is likely to present a short life-expectancy, and/or that is likely to develop metastases, and/or that is likely to relapse, and/or that is likely not to respond, or poorly respond, to treatments.

In a specific embodiment of the in vitro method for determining the prognosis of a patient suffering from early-stage and/or low-grade cancer, an expression level of ERRαhigher than a predetermined threshold indicates that the patient is likely to present a short life-expectancy. Indeed, the inventors have shown that a high ERRα expression correlated with risk of recurrence at an early stage of the disease in the pN0 subset and in the pN<3 lymph-node positive subset.

They have also shown that a higher proportion of patients with high ERRαexpression exhibited liver, lung, bone and soft tissues metastases compared to patients with low ERRα expression. Therefore, in another embodiment of the in vitro method for determining the prognosis of a patient suffering from early-stage and/or low-grade cancer, an expression level of ERRα higher than a predetermined threshold indicates that the patient is likely to develop metastases.

Further, since the inventors have reported a significant correlation between a high level of ERRα mRNA expression and a decrease in relapse free survival, an expression level of ERRα higher than a predetermined threshold indicates that the patient suffering from early-stage and/or low-grade cancer is likely to relapse.

On the other hand, in a patient suffering from early-stage and/or low-grade cancer, the measurement of no or low expression levels of ERRα is indicative of a good prognosis Therefore, in another embodiment of the in vitro method for determining cancer prognosis, an expression level of ERRα lower than a predetermined threshold is indicative of a good prognosis. In a specific embodiment, an expression level of ERRα lower than a predetermined threshold indicates that the patient is likely to present a long life-expectancy. Alternatively, an expression level of ERRα lower than a predetermined threshold indicates that the patient is not likely to develop metastases. Still alternatively, an expression level of ERRα lower than a predetermined threshold indicates that the patient is not likely to relapse.

The skilled person can easily determine such a predetermined threshold using methods well-known in the art. As used throughout the present specification, the term “predetermined threshold” refers to the median value of the expression level of ERRα in biological samples of a healthy individual.

For instance, real-time RT-PCR may be performed with primers specific for human ERRα, OPG and for housekeeping genes such as, for example, L32 and TBP. In particular, real-time RT-PCR may be performed with the following primers specific for human ERRα, OPG and for housekeeping genes L32 and TBP: L32: 5′-CAAGGAGCTGGAAGTGCTGC-3′,5′-CAGCTCTTTCCACGATGGCT-3′; TBP 5′-TGGTGTGCACAGGAGCAAG-3′,5′-TTCACATCACAGCTCCCCAC-3′; ERRα: 5′-ACCGAGAGATTGTGGTCACCA-3′,5′-CATCCACACGCTCTGCAGTACT-3′; and OPG: 5′-CACGACAACATATGTTCCGG-3′,5′-TGTCCAATGTGCCGCTGCACGC-3′. Real-time RT-PCR may be carried out by using SYBR Green (Qiagen,) on the LightCycler system (Roche) according to the manufacturer's instructions with an initial step for 10 minutes at 95° C. followed by 40 cycles of 20 seconds at 95° C., 15 seconds at Tm (L32: 62° C.; TBP: 67° C.; ERRα: 59° C.; OPG: 61° C.) and 10 seconds at 72° C. C_{T} may thus be obtained for L32, TBP and ERRα.

Data analysis may be carried out using the comparative Ct method: in real-time each replicate average genes C_{T} may be normalized to the average C_{T} of a housekeeping gene (for instance L32) by subtracting the average C_{T} of the housekeeping gene from each replicate. Average of CT of each gene may be calculated. Then values may be corrected depending of the efficiency of each couple of primers ((mean CT- Y-intercept)/slope=Q): ERRα: CT-20,1/-3,5; L32: CT-12,78/-3,46; TBP: CT-20,62/-3,559; OPG: CT-22,5/-3,77. Corrected value equal 10^{Q}. Then ERRα corrected value/L32 corrected value is called a. Then a is subtracted to the first value (first sample on the list) giving a value called b which is < or >2.65, the medium value of ERRα obtained by calculating the average of 254 values. The same calculation may be performed with TBP only. The average of the b values obtained with ERRα/L32 and ERRα/TBP may also be calculated and then compared with the criteria already established in the cohort.

In a specific embodiment of the in vitro method for determining the prognosis of a patient suffering from early-stage and/or low-grade cancer, a normalized expression level of ERRα, as calculated above using L32 as a housekeeping gene, of at least 2.65 is indicative of a poor cancer prognosis. Still more preferably, a normalized expression level of ERRα of at least 2.66, 2.68, 2.70, or 2.75 is indicative of a poor cancer prognosis.

The determination of the expression level of one or several of cancer markers different from ERRα may be advantageously used in combination with that of ERRα to determine cancer prognosis. The in vitro method for determining the prognosis of a patient suffering from early-stage and/or low-grade cancer may thus further comprise the step of measuring the expression level of at least one second cancer marker. Cancer markers useful for prognosing the outcome of the disease are well known by the skilled in the art. Such cancer markers comprise VEGF, OPG, Ki67, ER, progesterone receptor (PR), HER2, cyclin D1, cyclin E, p53, ARF, TBX2/3, BRCA-1, BRCA-2, ErbB oncogenes, transforming growth factor alpha (TGFα), and the multiple drug resistance (MDR) gene.

Moreover, the inventors have confirmed in vivo in mice the association between ERRα and VEGF and between ERRα and OPG, and the increased size and vascularity of primary tumors in mice inoculated with ERRα-overexpressing breast cancer cells. In particular, VEGF is a direct target gene of ERRα and the inventors have found that high expression of ERRα is associated with high expression of VEGF in the cohort. Therefore, in a preferred embodiment, the method comprises measuring the expression level of ERRα and of osteoprotegerin (OPG) and/or of the vascular endothelial growth factor (VEGF) in cancer cells of said patient.

As used throughout the present specification, the term “VEGF” refers to the human vascular endothelial growth factor. This term is meant to encompass any naturally occurring isoform of the VEGF protein, including the protein having an amino acid sequence of SEQ ID NO: 3, allelic variants thereof and splice variants thereof.

As used throughout the present specification, the term “OPG” refers to the human osteoprotegerin. This term is meant to encompass any naturally occurring isoform of the OPG protein, including the protein having an amino acid sequence of SEQ ID NO: 2, allelic variants thereof and splice variants thereof.

More preferably, an expression level of ERRα and of OPG and/or VEGF higher than a predetermined threshold is indicative of a poor prognosis.

Prognosis of Patients Suffering from Cancer-Derived Bone Metastases

Breast cancer invades locally and spreads initially through the regional lymph nodes, bloodstream, or both. Metastatic breast cancer may affect almost any organ in the body, most commonly, lungs, liver, bone, brain, and skin. Metastatic breast cancer frequently appears years or decades after initial diagnosis and treatment.

Bone metastases are a frequent complication of cancer, occurring in up to 70 percent of patients with advanced breast cancer.

The inventors have surprisingly reported the first evidence that ERRα is involved in development of bone metastases. The have shown that ERRα over-expression in cells from primary breast tumor decreases breast cancer cell-induced osteolytic lesion size, inhibits OCs formation and alters expression of a variety of OBs markers, including the main OCs inhibitor OPG in breast cancer cells. Overall, they have shown that ERRα has a protective and favourable role in osteolytic lesions and bone metastases development.

Therefore, while high ERRα expression levels is indicative of a poor prognosis in patients from early-stage and/or low-grade cancer, high ERRα expression levels in the primary tumor is indicative of a relatively good prognosis in patients suffering from breast cancer-derived bone metastases.

Therefore, a second aspect of the invention is an in vitro method for determining prognosis for a patient suffering from bone metastases, said method comprising:
a) providing or obtaining a biological sample comprising cells from the primary tumor of said patient (e.g. a breast cancer);
b) measuring the expression level of ERRα in said biological sample; and
c) optionally deducing from the result of step b) the prognosis of said patient.

In a specific embodiment, an expression level of ERRα lower than a predetermined threshold is indicative of a poor prognosis (e.g. the patient is likely to present a short life-expectancy, and/or to relapse, and/or not to respond, or poorly respond, to treatments).

The primary tumor preferably corresponds to a breast cancer. The bone metastases are the breast cancer-derived metastases.

More preferably, an expression level of ERRα of at most 2.65 is indicative of a poor bone metastases prognosis. Still more preferably, an expression level of ERRα of at most 2.64, 2.62, 2.60, or 2.55 is indicative of a poor bone metastases prognosis.

The above in vitro method for determining the prognosis of a patient suffering from cancer-derived bone metastases may further comprise the step of measuring the expression level of at least one second cancer marker such as, e.g., OPG, VEGF, Ki67, ER, progesterone receptor (PR), HER2, cyclin D1, cyclin E, p53, ARF, TBX2/3, BRCA-1, BRCA-2, ErbB oncogenes, transforming growth factor alpha (TGFα), and the multiple drug resistance (MDR) gene. In a preferred embodiment, the second cancer marker is OPG. Therefore, another aspect of the invention is an in vitro method for determining the prognosis of a patient suffering from cancer-derived bone metastases, said method comprising:

a) providing or obtaining a biological sample comprising cells from the primary tumor of said patient;
b) measuring the expression level of ERRα and osteoprotegerin (OPG) in said biological sample; and
c) optionally deducing from the result of step b) the prognosis of said patient.

In a specific embodiment, an expression level of ERRα and of OPG lower than a predetermined threshold is indicative of a poor prognosis for a patient suffering from cancer-derived bone metastases.

Biological Samples and Methods for Measuring ERRα Expression Level

In the above methods, the ERRα expression level may be measured using any method well-known in the art. The expression level may be measured at the nucleic acid level (e.g. through RT-PCR) or at the level of the protein (e.g. through immunofluorescence or flow cytometry).

For example, it may be determined by RT-PCR. Alternatively, it may be determined by immunofluorescence (immunocytochemistry or immunohistochemistry) or by western blot. Such methods are described in details in the examples.

When determined by RT-PCR, the expression level of ERRα may be normalized with the average of the expression level of housekeeping genes. For instance, the genes encoding the ribosomal protein L32 and the TATA-box binding protein TBP are housekeeping genes that may be used to normalize the expression level of ERRαmeasured by RT-PCR.

Immunofluorescence experiments may for example be performed using the antibodies that are commercialized by Santa cruz, and Epitomics, Burligame, Calif. The antibody preferably corresponds to a polyclonal antibody against human ERRαcommercialized by Santa Cruz, Tebu. Alternatively, the antibody may correspond to a monoclonal antibody against human ERRα, such as e.g. the mouse monoclonal antibody ERRα (1ERR87) sc-65715 raised against amino acids 1-76 of human ERRα, and commercialized by Santa Cruz.

In a specific embodiment, the expression level of ERRα is measured by measuring the level of ERRα mRNA. In another embodiment, the expression level of ERRα is measured by measuring the amount of ERRα protein.

The term “biological sample” refers to any type of biological sample. The skilled in the art will appreciate that the biological sample will depend on the tumor to be prognosed.

For example, in the frame of a breast cancer, the biological sample may e.g. correspond to breast tissue or to breast cells, most preferably epithelial breast cancer cells, which can for example be obtained by surgical excision or by biopsy. It can also be any biological fluid that may contain cancer cells. Therefore, the biological sample may correspond to a biological fluid such as blood, urine, semen or lymphatic fluid. The biological fluid may optionally be enriched for cancer tissue or cells.

Methods of Selecting Patients to be Treated by Preventive or Aggressive Therapies

The above methods for prognosing a patient may also be used for designing a treatment regimen, for monitoring the progression of the cancer, and/or for monitoring the response of the patient to a treatment.

When the above methods are used to monitor the progression of a disorder and/or to monitor the response to a treatment, it is repeated at least at two different points in time (e.g. before and after onset of a treatment).

When the above methods are used to design a treatment regimen, they further comprise the step of designing a treatment regimen based on the result of step (b). Typically, the patient is given a preventive treatment regimen if the prognosis is found to be poor.

The above methods can be used to decide how to monitor a patient. Indeed, as shown herein, high expression of ERRα is indicative of a poor cancer prognosis. Therefore, pN0 patients and/or ER+ patients who display a high expression level of ERRαshould be under very tight observation by their oncologist, although pN0 patients and/or ER+ patients were considered to have a good prognostic until now. That is to say, ERRαcan be used as a prevention marker.

More generally, patients who display a high expression level of ERRα in their cancer cells need to be treated by a preventive therapy. ERRα can thus be used as a marker for selecting the treatment regimen of a patient.

The invention is thus directed to an in vitro method for selecting a patient suffering from cancer, and/or from metastasis that is not a bone metastasis, suitable to be treated with a preventive therapy comprising the step of:

a) providing or obtaining a biological sample comprising cells from said cancer or from said metastasis;
b) measuring the expression level of ERRα in said biological sample; and
c) selecting the patient having an expression level of ERRα higher than a predetermined threshold.

The cancer preferably corresponds to breast cancer, and the metastasis to a breast cancer-derived metastasis.

In a specific embodiment, the above method further comprises the steps of:

b′) comparing the expression level of ERRα in said biological sample to a predetermined threshold; and
d) designing a treatment regimen comprising a preventive therapy if the expression level of ERRα in said biological sample is higher than the predetermined threshold.

The expression “metastasis that is not a bone metastasis” refers to any kind of metastasis that may appear following the development of primary cancer. The metastasis that is not a bone metastasis may for instance be a lung, liver, brain, or skin metastasis.

Cancer treatment options are related to a number of factors such as the stage of the cancer, the grade of the cancer, the invasiveness of the cancer, the ER status, the pN status, but also the overall prognosis of the cancer, the patient life-expectancy, the risk of metastasis, and the risk of relapses. Therefore, determining the prognostic of a patient may help selecting the treatment regimen of said patient.

Patients suffering from early-stage or low-grade cancer with good prognostic generally receive a light therapy. Such light therapy may only include careful watching, and behaviour modifications such as e.g. exercise and dietary changes, usually together with a breast-conserving surgery, such as e.g. local excision of the tumor, lumpectomy or partial mastectomy.

The patients overexpressing ERRα should be placed under close observation by their oncologist and/or receive a somewhat heavier treatment. Such a treatment is referred to as a “preventive treatment” herein.

In the field of the invention, a “preventive therapy” may refer to a preventive surgery, and/or a radiotherapy.

In another embodiment, the “preventive therapy” may refer to a systemic therapy. By systemic therapy is meant a therapy that is given thought the bloodstream, such as e.g. hormone therapy, chemotherapy and/or immunotherapy. Hormone therapy refers to the use of hormones and/or hormone antagonists, such as e.g. tamoxifen or raloxifene, in medical treatment. Chemotherapy refers to the treatment by chemicals such as antineoplastic drugs or a combination of these drugs. Antineoplastic drugs include e.g. cyclophosphamide, methotrexate, and 5-Fluorouracil. Immunotherapy refers to the treatment by induction, enhancement, or suppression of an immune response, using immuno-modulators such as e.g. trastuzumab.

Thus, in a specific embodiment, the “preventive therapy” may be a hormone therapy, a chemotherapy, an immunotherapy or any combination thereof. In a preferred embodiment, the “preventive therapy” refers to a combination of hormone therapy and chemotherapy.

In another embodiment, the “preventive therapy” may be a combination of surgery, optionally followed by radiotherapy, and of systemic therapy. Preferably, the “preventive therapy” refers to a combination surgery, radiotherapy, and hormone therapy. Alternatively, the “preventive therapy” refers to a combination surgery, radiotherapy, hormone therapy, and chemotherapy.

The invention also pertains to an in vitro method for selecting a patient suffering from bone metastases suitable to be treated with an aggressive therapy comprising:

a) providing or obtaining a biological sample comprising cells from the primary tumor;
b) measuring the expression level of ERRα in said biological sample; and
c) selecting the patient having an expression level of ERRα lower than a predetermined threshold.

The cancer preferably corresponds to breast cancer, and the metastasis to a breast cancer-derived metastasis.

In a specific embodiment, the above method further comprises the steps of:

b′) comparing the expression level of ERRα in said biological sample to a predetermined threshold; and
d) designing a treatment regimen comprising an aggressive therapy if the expression level of ERRα in said biological sample is lower than the predetermined threshold.

By “aggressive therapy” is meant a therapy adapted for treating aggressive cancers. Specifically, such aggressive therapies may induce side effects and do therefore not constitute the preferred treatment regimen in the case of a non-aggressive cancer. An aggressive chemotherapy typically corresponds to a combination chemotherapy carried out with high doses of drugs. The combination chemotherapy may for example comprise the administration of high doses of at least one compound selected from the group consisting of an alkylating agent, an antimetabolite, an antimitotic, a topoisomerase inhibitor, a hormonal therapy drug, a signaling inhibitor, an aromatase inhibitor, a differentiating agent, a monoclonal antibody, a biologic response modifier and an antiangiogenic agent. Thus combination chemotherapy may for example comprise the administration of at least one of the following anti-cancer agents (simultaneously or sequentially):
an alkylating agent such as Cyclophosphamide, Chlorambucil and Melphalan;
an antimetabolite such as Methotrexate, Cytarabine, Fludarabine, 6-Mercaptopurine and 5-Fluorouracil;
an antimitotic such as Vincristine, Paclitaxel (Taxol), Vinorelbine, Docetal and Abraxane;
a topoisomerase inhibitor such as Doxorubicin, Irinotecan, Platinum derivatives, Cisplatin, Carboplatin, Oxaliplatin;
a hormonal therapy drug such as Tamoxifen;
an aromatase inhibitor such as Bicalutamide, Anastrozole, Examestane and Letrozole;
a signaling inhibitor such as Imatinib (Gleevec), Gefitinib and Erlotinib;
a monoclonal antibody such as Rituximab, Trastuzumab (Herceptin) and Gemtuzumab ozogamicin;
a biologic response modifier such as Interferon-alpha;
a differentiating agent such as Tretinoin and Arsenic trioxide; and/or
an agent that block blood vessel formation (antiangiogenic agents) such as Bevicizumab, Serafinib and Sunitinib.
an agent that block osteoclast maturation and/or function such as bisphosphonate or denosumab

The aggressive therapy may also correspond to radiation therapy and/or surgery, or to a combination of chemotherapy with a radiation therapy and/or surgery.

Kits According to the Invention

The invention further discloses kits that are useful in the above methods. Such kits comprise means for detecting the amount and/or expression level of ERRα.

They can be used, e.g. for prognosing the outcome of a cancer in a patient, for designing a treatment regimen, for monitoring the progression of the cancer, and/or for monitoring the response of the individual to a drug (i.e. “drug monitoring”).

The kit may further comprise means for detecting the amount and/or expression level of other cancer markers ERRα, such as e.g. means for detecting the amount and/or expression level of OPG or VEGF. Optionally, the kit may further comprise means for detecting the amount and/or expression level of some housekeeping genes, such as e.g. L32 and TBP.

In a preferred embodiment, the kit according to the invention comprises, in addition to the means for detecting the amount and/or expression level of ERRα, a control sample indicative of the amount and/or expression level of ERRα in a patient suffering from cancer. Optionally, the kit according to the invention comprises in addition a control sample indicative of the amount and/or expression level of ERRα in a healthy individual.

The kits according to the invention may for example comprise, in addition to the means for detecting the amount and/or expression level of ERRα, one of (i) to (iii) below:
i. a positive control sample indicative of the amount and/or expression level of ERRα in a patient suffering from breast cancer;
ii. a negative control sample indicative of the amount and/or expression level of ERRα in a healthy individual;
iii. instructions for the use of said kit in prognosing breast cancer, in assessing the severity of a breast cancer and/or in prognosing breast cancer derived-metastases.

Such a kit may for example comprise (i) and (ii), (i) and (iii), (ii) and (iii), or (i), (ii) and (iii).

Means for detecting the amount and/or expression level of ERRα are well-known in the art. They include, e.g. reagents allowing the detection of ERRα mRNA by real-time quantitative-PCR, such as primers specific for ERRα. When the kit comprises means for real-time quantitative-PCR ERRα mRNA detection, the kit may further comprise a second reagent, labeled with a detectable compound, which binds to ERRα mRNA synthesized during the PCR, such as e.g. SYBER GREEN reagents.

Means for detecting the amount and/or expression level of ERRα may also include antibodies specifically binding to ERRα. Such means can be labeled with detectable compound such as fluorophores or radioactive compounds. For example, the probe or the antibody specifically binding to ERRα may be labeled with a detectable compound. Alternatively, when the kit comprises a antibody, the kit may further comprise a secondary antibody, labeled with a detectable compound, which binds to an unlabelled antibody specifically binding to ERRα.

The means for detecting the amount and/or expression level of ERRα may also include reagents such as e.g. reaction, hybridization and/or washing buffers. The means may be present, e.g., in vials or microtiter plates, or be attached to a solid support such as a microarray as can be the case for primers and probes.

The kit may for example include the anti-ERRα polyclonal antibody (Santa Cruz, Tebu) as a mean for detecting the amount and/or expression level of ERRα.

The invention further pertains to a kit for use in the diagnosis of patients suffering from early-stage or low-grade breast cancer as described herein, and in particular for use in the diagnosis of ER+ or pN0 patients suffering from early-stage or low-grade breast cancer.

### BRIEF DESCRIPTION OF THE SEQUENCES

SEQ ID NO: 1 shows the sequence of ERRα.

SEQ ID NO: 2 shows the sequence of OPG.

SEQ ID NO: 3 shows the sequence of VEGF.

SEQ ID NO: 4 shows the sequence of a primer specific for ERRα.

SEQ ID NO: 5 shows the sequence of a primer specific for ERRα.

SEQ ID NO: 6 shows the sequence of a primer specific for ERRα-ΔAF2-AD.

SEQ ID NO: 7 shows the sequence of a primer specific for human L32.

SEQ ID NO: 8 shows the sequence of a primer specific for human L32.

SEQ ID NO: 9 shows the sequence of a primer specific for ERRα.

SEQ ID NO: 10 shows the sequence of a primer specific for ERRα.

SEQ ID NO: 11 shows the sequence of a primer specific for TBP.

SEQ ID NO: 12 shows the sequence of a primer specific for TBP.

SEQ ID NO: 13 shows the sequence of a primer specific for human OPG.

SEQ ID NO: 14 shows the sequence of a primer specific for human OPG.

SEQ ID NO: 15 shows the sequence of a primer specific for human OPN.

SEQ ID NO: 16 shows the sequence of a primer specific for human OPN.

SEQ ID NO: 17 shows the sequence of a primer specific for human VEGF.

SEQ ID NO: 18 shows the sequence of a primer specific for human VEGF.

SEQ ID NO: 19 shows the sequence of a primer specific for human MMP1.

SEQ ID NO: 20 shows the sequence of a primer specific for human MMP1.

SEQ ID NO: 21 shows the sequence of a primer specific for human Runx2.

SEQ ID NO: 22 shows the sequence of a primer specific for human Runx2.

SEQ ID NO: 23 shows the sequence of a primer specific for human DKK1.

SEQ ID NO: 24 shows the sequence of a primer specific for human DKK1.

SEQ ID NO: 25 shows the sequence of a primer specific for human MMP13.

SEQ ID NO: 26 shows the sequence of a primer specific for human MMP13.

SEQ ID NO: 27 shows the sequence of a primer specific for human Noggin.

SEQ ID NO: 28 shows the sequence of a primer specific for human Noggin.

SEQ ID NO: 29 shows the sequence of a primer specific for human RANK.

SEQ ID NO: 30 shows the sequence of a primer specific for human RANK.

SEQ ID NO: 31 shows the sequence of a primer specific for human Osterix.

SEQ ID NO: 32 shows the sequence of a primer specific for human Osterix.

SEQ ID NO: 33 shows the sequence of a primer specific for human Cytochrome C.

SEQ ID NO: 34 shows the sequence of a primer specific for human Cytochrome C.

SEQ ID NO: 35 shows the sequence of a primer specific for human CDH11.

SEQ ID NO: 36 shows the sequence of a primer specific for human CDH11.

SEQ ID NO: 37 shows the sequence of a primer specific for human P21.

SEQ ID NO: 38 shows the sequence of a primer specific for human P21.

SEQ ID NO: 39 shows the sequence of a primer specific for human P27.

SEQ ID NO: 40 shows the sequence of a primer specific for human P27.

SEQ ID NO: 41 shows the sequence of a primer specific for human OCN.

SEQ ID NO: 42 shows the sequence of a primer specific for human OCN.

SEQ ID NO: 43 shows the sequence of a primer specific for murine L32.

SEQ ID NO: 44 shows the sequence of a primer specific for murine L32.

SEQ ID NO: 45 shows the sequence of a primer specific for murine OPN.

SEQ ID NO: 46 shows the sequence of a primer specific for murine OPN.

SEQ ID NO: 47 shows the sequence of a primer specific for murine ALP.

SEQ ID NO: 48 shows the sequence of a primer specific for murine ALP.

SEQ ID NO: 49 shows the sequence of a primer specific for murine BSP.

SEQ ID NO: 50 shows the sequence of a primer specific for murine BSP.

SEQ ID NO: 51 shows the sequence of a primer specific for murine OCN.

SEQ ID NO: 52 shows the sequence of a primer specific for murine OCN.

SEQ ID NO: 53 shows the sequence of a primer specific for murine RANKL.

SEQ ID NO: 54 shows the sequence of a primer specific for murine RANKL.

SEQ ID NO: 55 shows the sequence of a primer specific for murine OPG.

SEQ ID NO: 56 shows the sequence of a primer specific for murine OPG.

### BRIEF DESCRIPTION OF THE FIGURES

FIG. 1: ERRα is a bad prognostic marker and is expressed in bone metastases. Kaplan-Meier curves show correlation between high expression of ERRα, categorized with quartiles, and metastasis free survival in patients in (A) the whole population (N=251), and (B) the ER+ patients (N=209). Low≦50% quartile; high≧50%.

FIG. 2: ERRα is a bad prognostic marker and is expressed in bone metastases. Kaplan-Meier curves show correlation between high expression of ERRα, categorized with quartiles, and metastasis free survival in patients in (A) the pN0+ patients (N=115) and (B) the <3 lymph-node+ patients (N=198). Low≦50% quartile; high≧50%.

FIG. 3: Modulation of ERRα in BO2, a breast cancer cell line highly metastatic to bone. (A) Detection by real-time PCR of ERRα mRNA expression in several breast cancer cells lines and in BO2 cells. (B) Isolation, after stable transfection of the BO2 cell line, of three independent BO2-ERRαΔAF2 clones (ERRα dominant negative form), one clone BO2-ERRαWT and two controls (CT-1 and CT-2) BO2-CT (empty vector). ERRαexpression was assessed by real-time PCR on triplicate samples and normalized against that of the ribosomal protein gene L32 (ANOVA, p<0.0001). (C) VEGF and osteopontin (OPN) expression was increased in BO2-ERRαWT and decreased or not regulated in BO2-ERRαΔAF2 (ANOVA, p<0.0001 for VEGF and OPN in BO2-ERRαWT or BO2-ERRαΔAF2 versus BO2-CT (pool CT-1 and 2)).

FIG. 4: ERRα expression in BO2 cells regulates osteoclast formation. (A) In sections of tibiae taken from mice injected with BO2-ERRαWT-1, BO2-ERRαΔAF2 (pool 1, 2, 3) or BO2-CT (pool CT-1 and 2) cells shows decreased and increased surface of active osteoclast in BO2-ERRαWT-1 and BO2-ERRαΔAF2 (pool 1, 2, 3) respectively compared to CT (ANOVA, p<0.0001). (B) Primary mouse bone marrow cells were cultured in the presence of RANKL and M-CSF and treated or not with medium conditioned by BO2-ERRαWT-1, BO2-ERRαΔAF2 or BO2-CT cells. Fewer osteoclast formed in cultures treated with BO2-ERRαWT-1 conditioned medium, while more formed in cultures treated with BO2-ERRαΔAF2 conditioned medium, compared to cultures treated with BO2-CT (1, 2) conditioned medium (ANOVA, p<0.0001). (C) Conditioned medium obtained from parental BO2 cells treated with the ERRα inverse agonist XCT-790 increased osteoclast formation, mimicking the results obtained with BO2-ERRαΔAF2 conditioned medium (ANOVA, p<0.001).

FIGS. 5 to 7: ERRα regulates OBs markers in BO2 cell. Real-time PCR performed on RNA extracted from BO2-ERRαWT-1, BO2-ERRαΔAF2 (1,2,3) and BO2-CT (pool of 1 and 2) cells showed increased expression of Runx2 and osterix (OSX) in BO2-ERRαWT and decreased or no change in expression in BO2-ERRαΔAF2 (1,2,3) (ANOVA, p<0.0001 for Runx2 and OSX). Osteocalcin (OCN), a Runx2 and OSX target gene, was also regulated (ANOVA, p<0.0001). Osteoblast cadherin (cadherin 11 (CDH11)) was also increased by ERRα overexpression (ANOVA, p<0.0001). DKK1/Noggin and OPG, inhibitors of the osteoblast or osteoclast lineage respectively, were also regulated by changes in ERRα levels (ANOVA, p<0.0001). RANK was significantly affected by changes in ERRα levels (ANOVA, p=0.0367).

FIG. 8: Correlation of ERRα and OPG in BO2 cells and breast cancer patients. (A) ELISA quantification confirmed the increased secretion of OPG by BO2-ERRαWT compared to BO2-CT (pool) and BO2-ERRαΔAF2 (pool) cells (ANOVA, p=0.0064; p<0.01 CT versus WT-1 and WT-1 versus AF-2). (B) A significant correlation was also found between levels of ERRα mRNA and median values of OPG mRNA in the 251 patient cohort (ERRα 1st quartile and median OPG=2.03; ERRα 2nd-4-th quartile and median OPG=3.45).

FIG. 9: Correlation of ERRα and OPG in BO2 cells and breast cancer patients. (A) Kaplan-Meier curves show that ERRα+/OPG+ expression was associated with a decrease with metastasis free survival. (b) OPG alone was not associated with metastasis free survival.

FIG. 10: Stimulation of tumor progression and angiogenesis by ERRα in vivo. (A) BO2-ERRαWT, BO2-ERRαΔAF2 (pool) or BO2-CT (pool) cells were inoculated into the fat pad of NMRI nude mice. Tumor progression was followed by bioluminescence from day 5-66. Greater tumor expansion was observed in mice with BO2-ERRαWT-1 compared to BO2-ERRαΔAF2 (pool) or BO2-CT (pool) cells. (B-C) Weight of tumors dissected at endpoint (B) and VEGF expression within tumors (C) paralleled bioluminescence measurements and correlated with greater tumor vascularization respectively.

FIG. 11: Involvement of ERRα in BO2 invasion (A) Cell invasion was increased in BO2-ERRαWT-1 cells and decreased in BO2-ERRαΔAF2 cells versus BO2-CT cells (pool CT-1 and 2) (ANOVA p<0.0001). (B) Expression of MMP1 and MMP13 was regulated by ERRα level, as assessed by real-time PCR on triplicate samples; normalized against expression of the ribosomal protein gene L32 (ANOVA, p<0.0001).

FIG. 12: Inhibition of the osteoblastic lineage by BO2. Primary mouse calvaria cell cultures were treated from day 1-21 with medium conditioned by BO2 cell and used for osteoblast experiments. (A) Mineralized bone nodule formation was decreased when primary cells were treated with conditioned medium from any of the BO2 cells (compared with non-treated (NT) cells; see black asterisks); the decrease was less with BO2-ΔAF2 cell conditioned medium (compared with BO2-CT1-2; see surrounded asterisks) (ANOVA, p<0.0001 versus NT and versus CT). (B, C) Conditioned media stimulated the expression of OPN (early osteoblast marker; compared with non-treated (NT); see black asterisks and compared with BO2-CT1-2; see surrounded asterisks) (ANOVA, p<0.0001, versus NT and versus CT). Alkalin Phosphatase (ALP) was decreased (compared with non-treated (NT); see black asterisks) or slightly increased (compared with BO2-CT1-2; see surrounded asterisks) correlating with bone nodule number (ANOVA, p<0.0001 versus NT and versus CT). (D) OCN (late osteoblast marker) was dramatically decreased in all conditions (ANOVA, p<0.0001).

FIG. 13: Regulation of the RANKL/OPG ratio. OPG was not robustly or consistently regulated, while RANKL (receptor activator of nuclear factor kB ligand) was increased (compared with non-treated (NT); see black asterisks), with higher values in BO2-ΔAF2 clones (compared with BO2-CT1-2; see gray asterisks) (ANOVA, p<0.0001 versus NT and versus CT), leading to an increased RANKL/OPG ratio in BO2-AF2 clones.

FIG. 14: ERRα only marginally affects BO2 cell proliferation. BrdU incorporation was performed in BO2 clones. ERRα overexpresssion induced a slight decrease in cell proliferation in 5% serum, opposite to what was seen in BO2-ΔAF2 clones (pool of AF2-1, 2 and 3) (ANOVA, p<0.0001). Concomitantly, p21^{WAF1/CIP1} mRNA expression was increased in BO2-WT-1 but decreased or not regulated in BO2-ΔAF2 (1, 2 and 3) cells compared to CT (pool of CT-1 and 2 clones) (ANOVA, p<0.0001). No regulation of p27^{KIP1} expression was seen.

TABLE 1
ERRα expression in relation to the usual prognostic factors

| | | All patients | | | pN0 patients | | | pN+ patients | | |
|---|---|---|---|---|---|---|---|---|---|---|
| | | N = 251 | | | N = 115 | | | N = 136 | | |
| Characteristics | | n | Median | P-value | n | Median | P-value | n | Median | P-value |
| Menopausal Status | Pre | 105 | 2.68 | 0.425 | 48 | 2.59 | 0.020 | 79 | 3.10 | 0.229 |
| | Post | 146 | 2.59 | | 67 | 2.04 | | 57 | 2.72 | |
| Surgical Tumor size | <20 mm | 101 | 2.53 | | 58 | 2.15 | | 43 | 2.89 | |
| | ≧20 mm | 142 | 2.69 | 0.256 | 55 | 2.46 | 0.662 | 87 | 2.95 | 0.967 |
| | ND | 8 | | | | | | | | |
| Histological type | Ductal | 205 | 2.68 | | 91 | 2.32 | | 114 | 3.03 | |
| | Lobular | 37 | 2.09 | 0.026 | 16 | 1.71 | 0.208 | 21 | 2.21 | 0.058 |
| | Others | 9 | | | | | | | | |
| Histological grade* | 1 | 27 | 2.32 | | 14 | 2.22 | | 13 | 2.37 | |
| | 2 | 107 | 2.72 | | 53 | 2.41 | | 54 | 3.18 | |
| | 3 | 58 | 2.84 | 0.341 | 22 | 2.16 | 0.718 | 36 | 3.12 | 0.040 |
| | ND | 13 | | | | | | | | |
| Node status | Negative | 115 | 2.31 | | | | | | | |
| | 1-3 | 83 | 2.72 | | | | | 83 | 2.72 | |
| | >3 | 53 | 3.129 | <0.001 | | | | 53 | 3.12 | 0.439 |
| RE status | Negative | 42 | 3.17 | | 16 | 3.47 | | 26 | 3.17 | |
| | Positive | 209 | 2.53 | <0.001 | 99 | 2.12 | 0.003 | 110 | 2.78 | 0.106 |
| VEGF status** | Low | 92 | 2.35 | | 38 | 1.99 | | 54 | 2.62 | |
| | High | 93 | 3.08 | 0.002 | 42 | 2.57 | 0.018 | 51 | 3.23 | 0.010 |
| | ND | 66 | | | | | | | | |

*Histological grade defined only in ductal carcinoma;
**Low: <50% quartile; High: ≧50% quartile
P values correspond to Mann & Whitney test or Kruskall Wallis test (histological grade and node status)

TABLE 2
Effect of ERRα modulation on the formation and progression
of breast cancer osteolytic metastases and primary tumor in vivo.

| | | | ERRα | |
|---|---|---|---|---|
| | BO2-FRT Clones | CT | WT | ΔAF |
| Bone Metastases | Radiography | 10.946 ± 1.924 | 7. 11 ± 3.441* | 30.661 ± 7.166*** |
| | (mm²/animal) | (n = 9) | (n = 8) | (n = 9) |
| | Bone volume | 11.38 ± 1.08 | 15.13 ± 0.99* | 6.39 ± 0.415*** |
| | (BV/TV, %) | (n = 7) | (n = 6) | (n = 7) |
| | Tumor burden | 35.96 ± 4.21 | 21.04 ± 4.8* | 71.06 ± 3.15*** |
| | (TB/STV, %) | (n = 7) | (n = 6) | (n = 7) |
| | Osteolyse | 49.3 ± 3.4 | 21. ± 5.59** | 70.06 ± 6.04*** |
| | (Oc.S/BS, %) | (n = 8) | (n = 8) | (n = 9) |
| Tumorigenesis | BLU | 0.115 ± 0.03 | 1.67 ± 0.35*** | 0.34 ± 0.09 |
| (Fat Pad-Day 66) | (photons/sec 10⁶) | (n = 10) | (n = 10) | (n = 10) |
| | Volume | 104.45 ± 36 | 932 ± 152*** | 244 ± 67 |
| | (mm³/tumor) | (n = 10) | (n = 10) | (n = 10) |
| | Weight | 0.16 ± 0.03 | 0.98 ± 0.15*** | 0.32 ± 0.09 |
| | (g/tumor) | (n = 10) | (n = 10) | (n = 10) |

TABLE 3
Characteristics of the patient cohort.
Cox univariate analysis for metastasis-free survival.

| | All patients | | | pN0 patients | | | pN+ patients | | |
|---|---|---|---|---|---|---|---|---|---|
| | (n = 251) | | | (n = 115) | | | (n = 136) | | |
| Characteristics | HR | CI | P value | HR | CI | P value | HR | CI | P value |
| Menopausal Status | | | | | | | | | |
| Post | 1.00 | | | 1.00 | | | 1.00 | | |
| Pre | 1.49 | 0.87-2.54 | 0.145 | 2.66 | 0.77-9.12 | 0.121 | 1.29 | 0.71-2.35 | 0.406 |
| Surgical Tumor size | | | | | | | | | |
| <20 mm | 1.00 | | | 1.00 | | | 1.00 | | |
| ≧20 mm | 2.50 | 1.31-4.76 | 0.006 | 1.19 | 0.36-3.96 | 0.780 | 2.70 | 1.20-6.09 | 0.016 |
| Histological type | | | | | | | | | |
| Lobular | 1.00 | | | 1.00 | | | 1.00 | | |
| Ductal | 0.85 | 0.43-1.70 | 0.653 | 1.12 | 0.24-5.29 | 0.884 | 0.78 | 0.36-1.67 | 0.518 |
| Histological grade* | | | | | | | | | |
| 1 | 1.00 | | | 1.00 | | | 1.00 | | |
| 2 | 5.31 | 0.71-39.54 | | 0.73 | 0.08-6.70 | | | | |
| 3 | 10.44 | 1.40-77.83 | 0.014 | 1.50 | 0.16-13.83 | 0.597 | | | NA** |
| Node status | | | | | | | | | |
| Negative | 1.00 | | | | | | | | |
| 1-3 | 1.61 | 0.72-3.58 | | | | | 1.00 | | |
| >3 | 6.62 | 3.22-13.64 | <0.001 | | | | 4.04 | 2.15-7.58 | <0.001 |
| RE status | | | | | | | | | |
| Positive | 1.00 | | | 1.00 | | | 1.00 | | |
| Negative | 1.96 | 1.05-3.67 | 0.036 | 0.71 | 0.09-5.67 | 0.750 | 2.14 | 1.10-4.19 | 0.026 |
| VEGF status | | | | | | | | | |
| Low*** | 1.00 | | | 1.00 | | | 1.00 | | |
| High | 2.68 | 1.38-5.19 | 0.004 | 2.59 | 0.5-13.42 | 0.257 | 2.84 | 1.38-5.84 | 0.005 |
| ERRalpha status | | | | | | | | | |
| Low*** | 1.00 | | | 1.00 | | | 1.00 | | |
| High | 1.80 | 1.04-3.13 | 0.037 | 3.61 | 1.06-12.38 | 0.041 | 1.13 | 0.61-2.10 | 0.699 |

HR = Hazard Ratio;
CI = Confidence Interval;
*Histological grade defined only in ductal carcinoma;
**No events in histological grade 1 tumor subset;
***Low < 2nd Quartile, High > 2nd Quartile
P values correspond to Cox regression model.

TABLE 4
Mouse primers and using conditions.

| Gene | Primers | PCR cycles | T (° C.) |
|---|---|---|---|
| L32 | CAAGGAGCTGGAGGTGCTGC | 30 | 59 |
| | CTGCTCTTTCTACAATGGC | | |
| OPN | GGTGATAGCTTGGCTTATGG | 30 | 59 |
| | GGCATGCTCAGAAGCTGGG | | |
| ALP | CCCGAATCCTTAAGGGCCAG | 35 | 59 |
| | TATGCGATGTCCTTGCAGC | | |
| BSP | TGCCTACTTTTATCCTCCTCTG | 35 | 59 |
| | ACCCGAGAGTGTGGAAAGTG | | |
| OCN | TGACAAAGCCTTCATGTCCA | 35 | 59 |
| | GAGAGGACAGGGAGGATCAA | | |
| RANKL | GTGGTCTGCAGGATCGCTCTG | 40 | 63 |
| | CGCTGGGCCACATCCAACC | | |
| OPG | TGTGTGACAAATGTGCTCC | 35 | 59 |
| | GTCTCACCTGAGAAGAACCC | | |

TABLE 5
Human primers and using conditions.

| | | PCR | T |
|---|---|---|---|
| Gene | Primers | cycles | (° C.) |
| L32 | CAAGGAGCTGGAAGTGCTGC | 25 | 62 |
| | CAGCTCTTTCCACGATGGCT | | |
| OPN | CGCCGACCAAGGAAAACTCA | 38 | 64 |
| | AACGGGGATGGCCTTGTATG | | |
| OPG | CACGACAACATATGTTCCGG | 40 | 61 |
| | TGTCCAATGTGCCGCTGCACGC | | |
| VEGF | AGGAGGAGGGCAGAATCA | 35 | 54 |
| | TCTATCTTTCTTTGGTCTGCATT | | |
| MMP1 | CCAGGCCCAGGTATTGGAGGGG | 40 | 61 |
| | GGCCGAGTTCATGAGCCGC | | |
| Runx2 | GGAGTGGACGAGGCAAGAGTTT | 40 | 63 |
| | AGCTTCTGTCTGTGCCTTCTGG | | |
| DKK1 | TAGCACCTTGGATGGGTATT | 25 | 58 |
| | ATCCTGAGGCACAGTCTGAT | | |
| MMP13 | CTTAGAGGTGACTGGCAAAC | 40 | 59 |
| | GCCCATCAAATGGGTAGAAG | | |
| Noggin | GAGCCGCCTCCGGAGAGAGACG | 40 | 60 |
| | TAGGGTCTGGGTGTTCGATG | | |
| RANK | GGGCAGATGTCTGCACAG | 35 | 68 |
| | CTTGAAGTTCATCACCTGCCC | | |
| Osterix | CCTGGCTCCTTGGGACCCGT | 40 | 59 |
| | ATTTGCTGCACGCTGCCGTC | | |
| Cytochrome C | GACCTCAGCTACATCGTGCG | 42 | 59 |
| | CGTGCTCTGGCTCAGATGCC | | |
| CDH11 | GGCAGGTGCTACAGCGCTCC | 30 | 59 |
| | TCCCTGTCCACCGCCTGAGC | | |
| P21/^{cip1} | GAGTTGGGAGGAGGCAGGCG | 25 | 59 |
| | GGACTGCAGGCTTCCTGTG | | |
| p27/^{kip1} | GGCTAACTCTGAGGACACG | 25 | 59 |
| | GATGTATCTGATAAACAAG | | |
| OCN | CCACCGAGACACCATGAGAGCCC | 40 | 62 |
| | GGGGACTGGGGCTCCCAGC | | |

### EXAMPLES

### Example 1

### Materials and Methods

Ethics Statement

The mice used in this study were handled according to the rules of Décret No. 87-848 du 19/10/1987, Paris. The experimental protocols were reviewed and approved by the Institutional Animal Care and Use Committee of the Université Claude Bernard Lyon-1 (Lyon, France). Mice were routinely inspected by the attending veterinarian to ensure continued compliance with the proposed protocols. BALB/c and NMRI mice were housed under barrier conditions in laminar flow isolated hoods. Animals bearing tumor xenografts were carefully monitored for established signs of distress and discomfort and were humanely euthanized. Studies involving human primary breast tumors were performed according to the principles embodied in the Declaration of Helsinki. Samples were included anonymously in this study. All human experiments were approved by the Experimental Review Board from the Laennec School of Medicine.

Breast Cancer Tissue Specimens

The autopsy files of the Department of Pathology (Pr. J. Boniver, Centre Hospitalier Universitaire of Liège, Belgium) were searched for diagnosis of disseminated breast cancer with histologically-proven bone metastases during the period from 1991 to 1998. Slides were retrieved, and clinical history was obtained. Two breast cancer patients who died with disseminated disease, including bone metastases, were selected for immunohistochemistry. Soft tissue metastases were fixed with formalin, dehydrated, and paraffin-embedded. Formalin-fixed, bone specimens were decalcified with a solution of ethylenediaminetetraacetic acid (EDTA) and hydrochloric acid (Decalcifier II, Surgipath Europe Ltd., Labonord, Waregem, Belgium) or with a solution of formalin (20%) containing 5% (v/v) nitric acid. Paraffin-embedded tissue blocks were sectioned at 5 μm. Slides were then processed for immunostaining.

### Breast Cancer Cohort of Patients

Patients were selected according to the following criteria: primary breast tumor without inflammatory features, no previous treatment. Patient tumors were provided by three medical centers (Centre Hospitalier Régional Annecy, Chirurgie Oncologique Centre Hospitalier Universitaire Lyon-Sud, and Clinique Mutualiste Saint Etienne, France) in which patients were included between October 1994-2001. Breast cancer tissue biopsy were obtained by surgery, selected by the pathologist and immediately stored in liquid nitrogen until processing. The biopsies were pulverized using a MikroDismembrator (B. Braun Biotech International) and total RNA was extracted using Trizol Reagent (Sigma). To remove any genomic DNA contamination, total RNA was treated with RNAse-free DNAse I and purified using RNeasy microcolumns (Qiagen). RNA quality was verified using an Agilent Bioanalyser 2100 (Agilent Technologies). Real-time RT-PCR was performed (see RT-PCR section)

Cell Lines and Transfection

MDA-BO2-FRT (BO2) cells and stably transfected clonal derivatives were cultured in complete DMEM (Invitrogen), 10% fetal bovine serum (FBS, Perbio) and 1% penicillin/streptomycin (Invitrogen) at 37° C. in a 5% CO2 incubator. Human T47D, HS-578T and MDA-231 breast cancer cell lines were obtained from the American Type Culture Collection. Characteristics of MDA-MB-231/BO2-FRT (BO2) breast cancer cells were previously described (Peyruchaud et al, 2003, Early detection of bone metastases in a murine model using fluorescent human breast cancer cells: application to the use of the bisphosphonate zoledronic acid in the treatment of osteolytic lesions; J Bone Miner Res; 16(11): 2027-34). To avoid potential effects of different insertion sites, a pcDNA5/FRT vector (Invitrogen) was used to obtain the stable BO2-ERRαWT, BO2-ERRαΔAF2, and BO2 (CT) cell lines. Human ERRα cDNA (WT and ΔAF2-AD) was obtained from mRNA extracted from BO2-FRT cells, by using RT-PCR with specific primers ((NM_{—}004451.3): ERRα upstream (177 bp): GGG AAG CTT AGC GCC ATG TCC AGC CAG; ERRα downstream (WT) (177-1461 bp): GGG GGA TCC CCA CCC CTT GCC TCA GTC C; ERRα downstream (ΔAF2-AD): GGG GGA TCC TCA TGT CTG GCG GAG GAG (177-1350 bp; helix11-12 deletion (32 amino acids)). Amplimers were sequenced for verification. The pcDNA5/FRT/ERRα-WT and pcDNA5/FRT/ERRα-ΔAF2-AD constructs were co-transfected using Transfast (Promega) with the plasmid POG44 (Invitrogen) conferring the specific integration of ERRα-WT-1 and ERRα-ΔAF2-AD into the FRT site present in the BO2 cells. For clonal selection, cells were cultured for 4 weeks in the presence of hygromycin (20 mg/ml) (Invitrogen). One ERRα-WT-1, three ERRα-ΔAF2 and two BO2-CT were used for the present study. Conditioned medium from BO2-CT, BO2-ERRα-WT-1, BO2-ERRαΔAF2 and from BO2 treated with the inverse-agonist XCT-790 at 5.10⁻⁷M (Sigma) were obtained after 48 h in a-MEM supplemented with 0.5% of serum, then filter sterilized and proteins quantified in order to use equal concentration of proteins for each conditions (25 mg).

Animal Studies

Tumor fat pad experiments were performed using BO2-ERRαWT-1, BO2-ERRαΔAF2 (pool of AF2-1, 2 and 3 clones) and BO2 (CT1/2) cell lines (10⁶ cells in 50 ml of PBS) injected into the fat pad of the 4^{th} mammary gland of female 4-week-old NMRI nude mice (Charles River). Tumor progression was followed by bioluminescence (NightOwl, Berthold), then tumor size and weight were determined at 66 days after sacrifice.

Bone metastases experiments using BO2-ERRαWT-1, BO2-ERRαΔAF2 (pool of AF2-1, 2 and 3 clones) and BO2(CT1/2) cell lines were performed in 4-week-old BALB/c nude mice as previously described (LeGall et al, 2008; A cathepsin K inhibitor reduces breast cancer induced osteolysis and skeletaltumor burden. Cancer Res. 2007 Oct. 15; 67(20):9894-902). Cells were suspended at a density of 5×10⁵ in 100 ml of PBS and inoculated intravenously into animals. Radiographs (LifeRay HM Plus, Ferrania) of animals were taken at 35 days after inoculation using a cabinet X-ray system (MX-20; Faxitron X-ray Corporation). Animals were sacrificed and hind limbs were collected for histology and histomorphometrics analyses. Three-dimensional reconstructions of tibiae were performed using microcomputed tomography (microCT) (scanner CTan and CTvolsoftware and Skyscan1076). The area of osteolytic lesions was measured using the computerized image analysis system MorphoExpert (Exploranova). The extent of bone destruction for each animal was expressed in mm².

Bone Histomorphometry and Histology

Hind limbs from animals were fixed, decalcified with 15% EDTA and embedded in paraffin. Five mm sections were stained with Goldner's Trichrome and processed for histomorphometric analyses to calculate the BV/TV ratio (bone volume/tissue volume) and the TB/TV ratio (tumor burden/tissue volume). The in situ detection of osteoclasts was carried out on sections of bone tissue with metastases using the tartarte-resistant acid phosphatase (TRAP) activity kit assay (Sigma). The resorption surface (Oc.S/BS) was calculated as the ratio of TRAP-positive trabecular bone surface (Oc.S) to the total bone surface (BS) using the computerized image analysis system MorphoExpery (Exploranova).

Osteoclastogenesis Assay

Bone marrow cells from 6-week-old OF1 male mice were cultured for 7 days in differentiation medium: a-MEM medium containing 10% fetal calf serum (Invitrogen), 20 ng/mL of M-CSF (R&D Systems) and 200 ng/mL of soluble recombinant RANKL (David et al, 2010, Cancer cell expression of autotaxin controls bone metastasis formation in mouse through lysophosphatidic acid-dependent activation of osteoclasts. PLoS One 5(3)). Cells were continuously (day 1 to day 7) exposed to conditioned medium extracted (25 mg proteins for each conditions) from BO2-CT, BO2-ERRαWT and BO2-ERRαΔAF2 cells. After 7 days, mature multinucleated osteoclasts (OCs) were stained for TRAP activity (Sigma-Aldrich), following the manufacturer's instructions. Multinucleated TRAP-positive cells containing three or more nuclei were counted as OCs.

Osteoblastogenesis Assay

Cells were enzymatically isolated from the calvaria of 3-day-old OF-1 mice by sequential digestion with collagenase, as described previously (Bellows, 1986, Mineralized bone nodules formed in vitro from enzymatically released rat calvaria cell populations, Calcif Tissue Int 38:143-154). Cells obtained from the last four of the five digestion steps (populations II-V) were plated into 24-well plates at 2×10⁴ cells/well. After 24 hours incubation, the medium was changed and supplemented with 50 mg/ml ascorbic acid (Sigma-Aldrich). 10 mM sodium β-glycerophosphate (Sigma-Aldrich) was added for 1 week at the end of the culture period. Mouse calvaria cells were continuously (day 1 to day 15) exposed to conditioned medium (25 mg proteins for each conditions) extracted from BO2-CT, BO2-ERRα-WT-1 and BO2-ERRα-ΔAF2 clones. For quantification of bone formation, wells were fixed and stained with von Kossa and for ALP and bone nodules were counted on a grid. Results are plotted as the mean number of nodules±SD of three wells for controls and each condition (BO2-CT, BO2-ERRα-WT and BO2-ERRα-ΔAF2) and are representative of three independent experiments.

Immunofluorescence

BO2 cultures were fixed in culture wells with 3.7% paraformaldehyde (Sigma) in PBS for 10 min and permeabilized with 0.2% Triton X-100 in PBS. Immunodetection was performed using a goat polyclonal antibody against human ERRα (Santa Cruz, Tebu) at a dilution of 1/60 overnight at 4° C. and the secondary antibody (FITC-conjugated donkey anti-goat) at a dilution of 1/300 for 1 hour (Rockland, Tebu-bio). The distribution of F-actin was visualized after incubation of permeabilized cells for 50 min at room temperature with phalloidin (Molecular Probes) according to the manufacturer's instructions. Cells were observed using a LMS510 laser scanning confocal microscope (Zeiss, Le Pecq, France) with a 63× (numerical aperture 1.4) Plan Neo Fluor objective. To prevent contamination between fluorochromes, each channel was imaged sequentially, using the multi-track recording module, before merging. Z-cut pictures were obtained using Zeiss LSM 510 software.

Immunoblotting

Cell proteins were extracted, separated in 4-12% SDS-PAGE (Invitrogen), then transferred to nitrocellulose membranes (Millipore) using a semidry system. Immunodetection was performed using a goat polyclonal antibody against human ERRα(Santa Cruz) at a dilution of 1/400 overnight at 4° C. and the secondary antibody (HRP-conjugated donkey anti-goat) at a dilution of 1/4000 (Santa Cruz). For evaluating protein loading, a mouse polyclonal antibody against human α-tubulin (Sigma-Aldrich) and HRP-conjugated donkey anti-mouse (Amersham) was used at a dilution of 1/20000. An ECL kit (Perkin Elmer) was used for detection.

immunocytochemistry

Hind limbs were fixed and embedded in paraffin. Five mm sections were subjected to immunohistochemistry using a goat polyclonal antibody against human ERRα (Santa Cruz, Tebu) and a rabbit polyclonal antibody against human OPG (Abbiotec). Sections were deparaffinized in methylcyclohexane, hydrated, then treated with a peroxidase blocking reagent (Dako). Sections were incubated with normal calf serum for 1 hour, then treated with hydrogen peroxide and incubated overnight at 4° C. with primary antibody to ERRα and OPG (dilution: 1/50). Sections were incubated with secondary antibody HRP-conjugated donkey antigoat and anti-rabbit respectively (Amersham) (dilution 1/300) for 1 hour. After washing, the sections were revealed by 3,3′-diaminobenzidine (Dako). Counterstaining was performed using Mayer's hematoxylin (Merck).

Real Time RT-PCR

Total RNA was extracted with Trizol reagent (Sigma) from cancer cells, OBs, and OCs. Samples of total RNA (1 mg) were reverse-transcribed using random hexamer (Promega) and the first strand synthesis kit of Superscript™ II (Invitrogen). Real-time RT-PCR was performed on a Roche Lightcycler Module (Roche) with primers specific for human L32 (101 bp): 5′-CAAGGAGCTGGAAGTGCTGC-3′,5′-CAGCTCTTTCCACGATGGCT-3′; TBP (138 bp) 5′-TGGTGTGCACAGGAGCAAG-3′,5′-TTCACATCACAGCTCCCCAC-3′; ERRα (101 bp): 5′-ACCGAGAGATTGTGGTCACCA-3′, 5′-CATCCACACGCTCTGCAGTACT-3′; see Table 4 and 5. Real-time RT-PCR was carried out by using (SYBR Green; Qiagen,) on the LightCycler system on (Roche) according to the manufacturer's instructions with an initial step for 10 min at 95° C. followed by 40 cycles of 20 sec at 95° C., 15 sec at respective Tm and 10 sec at 72° C. The inventors verified that a single peak was obtained for each product using the Roche software. Amplimers were all normalized to corresponding L32 values. Data analysis was carried out using the comparative Ct method: in real-time each replicate average genes C_{T} was normalized to the average C_{T} of L32 by subtracting the average C_{T} of L32 from each replicate to give the ΔCT. Results are expressed as Log^{-2 ΔΔCT} with DDCT equivalent to the ΔC_{T} of the genes in BO2-ERRα-WT-1 or BO2-ERRα-ΔAF2 or treated OBs and OCs subtracting to the ΔC_{T} of the endogenous control (BO2-CT(1/2), non-treated OBs and OCs respectively).

Real-time RT-PCR on breast cancer tissue biopsy mRNA was performed using SYBR green (Invitrogen) in 96-well plates on a Mastercycler^{R}EP system (Realplex2, Eppendorf) according to the manufacturer's instructions and with primers specific for human L32, TBP, ERRα and OPG (see sequences page 8). ERRα and OPG expression were normalized with the average of the genes expression encoding the ribosomal protein L32 and the TATA-box binding protein TBP.

Cell Proliferation Assay

Experiments were carried out in conditions described previously (David et al, 2010, Cancer cell expression of autotaxin controls bone metastasis formation in mouse through lysophosphatidic acid-dependent activation of osteoclasts. PLoS One. 5(3)). BO2-CT(1/2), BO2-FRT-ERRα-WT and BO2-FRT-ERRα-ΔAF2 (pool of AF2-1, 2 and 3 clones) were plated in 48-well plates and cultured in complete medium for 24 h. Cells were then synchronized in serum-free medium for 24 h. Cell proliferation was evaluated following BrdU incorporation for 7 h in serum-containing medium and the use of the cell proliferation ELISA kit (Roche).

Cell Invasion Assay

Invasion assays were carried out using Bio-Coat migration chambers (Becton Dickinson) with 8 mm filters coated with Matrigel as described previously (Boissier et al, 2000, Bisphosphonates inhibit breast and prostate carcinoma cell invasion, an early event in the formation of bone metastases. Cancer Res. 60(11):2949-54). BO2 cells (5×10⁴) were plated in the upper chambers and the chemoattractant (10% FBS) in the lower chambers. After 24 h at 37° C. in 5% CO2 incubator, cells that had migrated through the filters were fixed and stained. Cells were counted from 12 random microscopic fields (200× magnification). All experiments were run in triplicate and invasion was expressed in cells/mm².

OPG ELISA

Conditioned medium obtained from BO2-CT(1/2), BO2-ERRα-WT-1 and BO2-FRT-ERRα-ΔAF2 (pool of AF2-1, 2 and 3 clones) were diluted following the manufacturer's instructions and OPG concentration was evaluated using the ELISA kit (RayBiotech).

Statistical Analysis

Data were analyzed statistically by one way analysis of variance (ANOVA) followed by post hoc t-tests to assess the differences between groups. The non parametric Mann-Whitney test or Kruskall-Wallis test were used for the clinical data. Results of p<0.05 were considered significant.

### Example 2

### ERRα mRNA and Protein Expression in Breast Cancer Patients and Bone Metastases

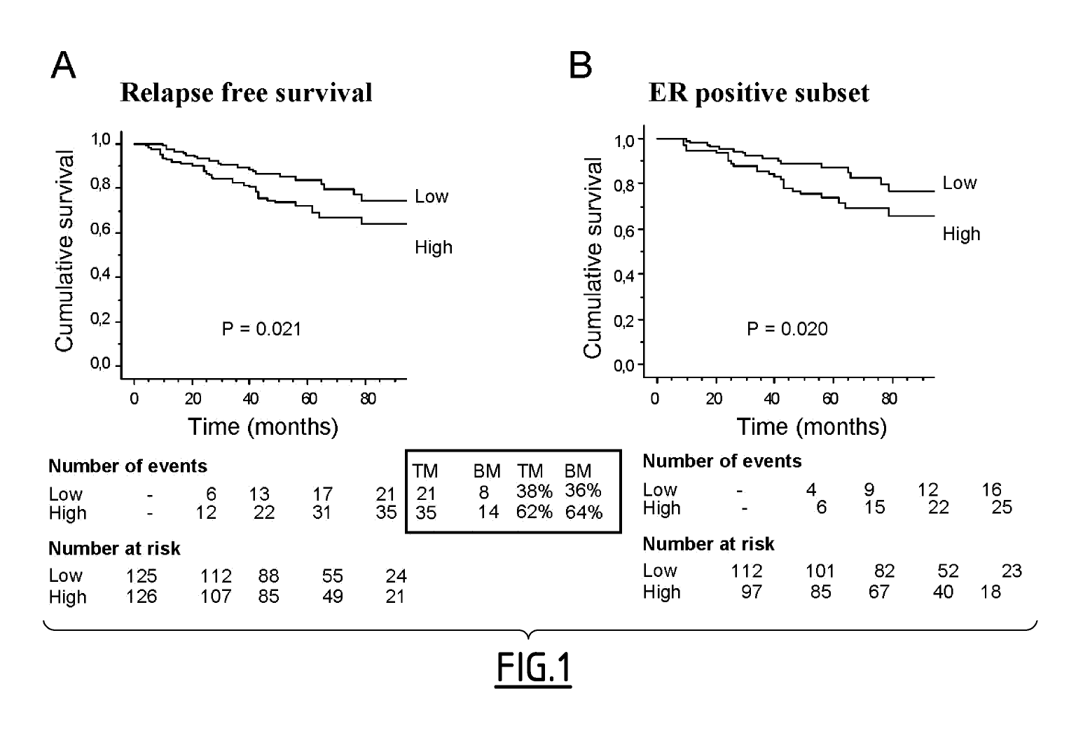

The inventors analyzed ERRα mRNA expression by real-time RT-PCR in a cohort of 251 breast tumor biopsies (Table 3). The median value of ERRα expression in relation with the clinicopathological parameters examined in this study showed an association between ERRα expression and clinical outcome (Table 1). A significant association was detected in all patients (N=251) between ERRα expression and histological type, node status, ERs (radioligand method) and VEGF (p=0.026, p<0.001, p<0.001 and p=0.002 respectively) (Table 1). Moreover, overall survival curves showed a significant correlation between a high level of ERRα mRNA expression and a decrease in relapse free survival (N=251) (p=0.021, log-rank test) (FIG. 1A). Sixty-two percent of patients with high ERRαexpression exhibited liver, lung, bone and soft tissues (TM) metastases compared to 38% with low ERRα (FIG. 1A). This paralleled the frequencies seen in patients who had developed “only” bone metastases (BM), i.e 64% (high ERRα) and 36% (low ERRα) (FIG. 1A).

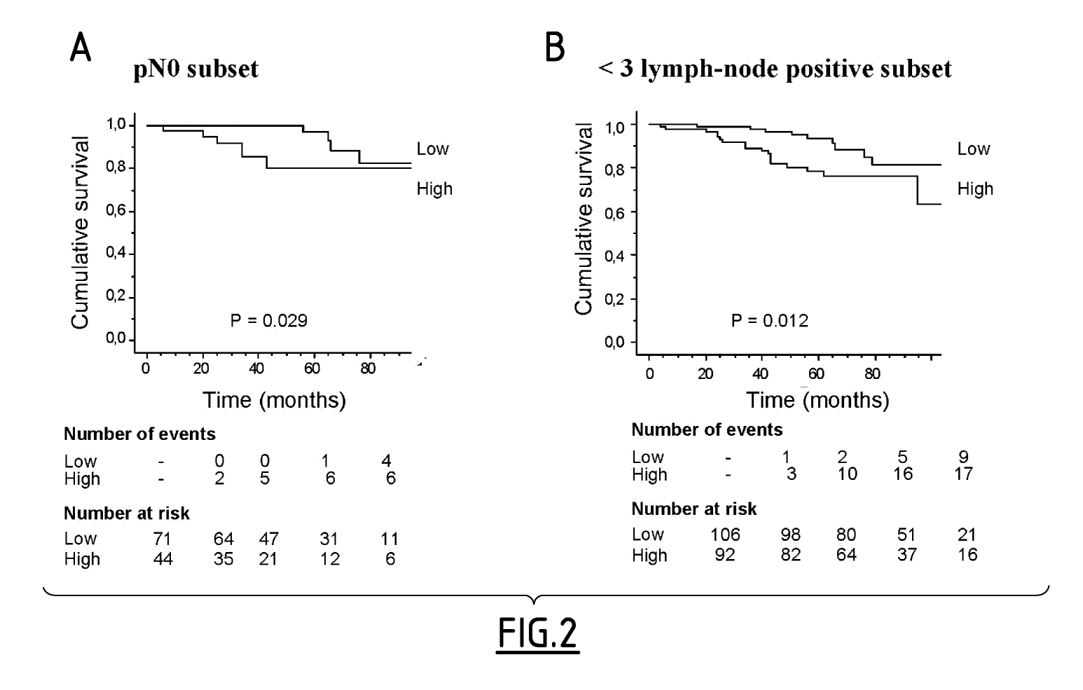

In particular, in the ER-positive group (N=209), high ERRα expression was also associated with a decrease in survival (P=0.02; log-rank test) (FIG. 1B). Notably, high ERRα expression also correlated with risk of recurrence at an early stage of the disease in the pN0 subset (N=115; P=0.029; log-rank test) and in the pN<3 lymph-node positive subset (N=198; P=0.012; log-rank test) (FIG. 2A, B). The median value of ERRα in the pN0 subset was significantly associated with ERs and VEGF (P=0.003 and P=0.018) but not with the histological type, although a trend was present in the latter (Table 1).

Finally, as previously described (Suzuki et al, 2004, Estrogen-related receptor alpha in human breast carcinoma as a potent prognostic factor, Cancer Res, 64 (13): 4670-6), ERRα protein was present in the cytoplasm and the nucleus of in situ and invasive breast carcinoma cells but was not detected in normal breast epithelium. In addition, ERRα was clearly present in breast cancer cells that had metastasized to bone. ERRα was also detected in osteocytes embedded in the bone matrix. Given these expression profiles, the inventors next asked whether ERRα is involved in breast metastases formation.

### Example 3

### ERRα Regulates OPN and VEGF Expressions in Breast Cancer Cell Line MDA-BO2 Cells

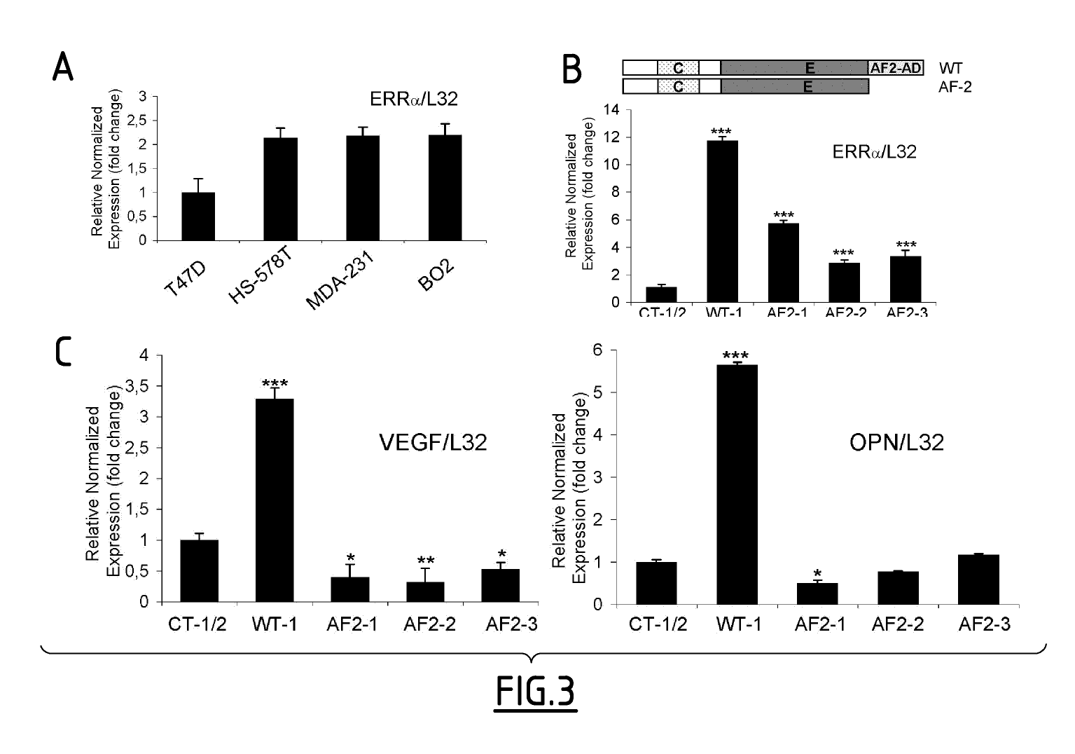

To assess whether ERRα is involved in bone metastases formation, the inventors used MDA-BO2-FRT (BO2) cells, a line that is highly and only metastatic to bone that derived from the MDA-231 human breast cancer cell line. Real-time RT-PCR revealed that ERRα mRNA is expressed in BO2 cells at a similar level to that in other ERα negative human breast cancer cell lines tested, HS-578T and MDA-231. Confirming the clinical datas ERRα was found less expressed in T47D cells, an ERα positive cell line (FIG. 3A). ERRα protein was also seen in the nucleus and cytoplasm of BO2 cells in vitro and in vivo in the bone metastases present after 30 days post intravenous inoculation. ERRα was also detected in chondrocytes in the growth plate and in osteocytes and osteoblasts.

To establish a functional role for ERRα in bone metastases development, the inventors next used a WT and a truncated version of ERRα lacking the co-activator binding domain AF2, ERRαΔAF2, which acts as a dominant negative form (FIG. 3B). Constructs of human ERRαWT and human ERRαΔAF2 were stably transfected into the FRT site present in the BO2 cells, conferring the specific integration of both constructs into the BO2 cells. BO2 cells were also stably transfected with the vector alone, which served as control (CT). Three independent BO2-ERRαΔAF2 (1, 2, 3), one BO2-ERRαWT and two vector alone BO2-CT clones were obtained, named AF2-1, AF2-2, AF2-3, WT-1, CT-1 and CT-2, respectively. Total ERRα mRNA expression was quantified by real-time PCR and found to be 12× for WT-1 versus CT-1/2 (pool of CT-1 and 2 clones) and 4-6× for AF2-1, AF2-2 and AF2-3 (FIG. 3B). Western blotting detected a protein of approximately 50 kD for ERRα protein in CT1-2 and WT-1 cells. As expected, ERRαexpression was higher in WT-1 and, AF2-1, AF2-2, AF2-3 cells than in CT-1 and CT-2 cells; the presence of a band of slightly lower MW in AF2-1, AF2-2, AF2-3 cells corresponds well with the truncation of the AF2 domain (42 aa). Also as expected, the expression level of the ERRα target genes VEGF and OPN was significantly increased in WT-1 clones and reduced or not affected in all three AF2-1, AF2-2, AF2-3 clones in comparison to CT-1/2 cells (FIG. 3C), confirming the increased activity and the dominant-negative functions of the WT and the truncated ERRαΔAF2 constructs respectively.

### Example 4

### ERRα Inhibits Osteolytic Bone Lesions In Vivo

To assess the involvement of ERRα in bone metastases formation, the inventors intravenously inoculated CT (pool of CT-1 and 2 clones), WT-1 and AF2 (pool of AF2-1, 2 and 3 clones) cells into BALB/c nude female mice. Thirty-five days after injection, radiographic analyses revealed that animals bearing WT-1 and AF2 cells exhibited a 1.5× decrease and 3× increase respectively in the extent of osteolytic lesions compared to CT cells (Table 2). The inhibitory effect of ERRα on cancer-induced bone destruction was confirmed using three-dimensional microCT reconstruction, histology and histomorphometric analyses (bone volume (BV/TV); skeletal tumor burden (TB/STV); Table 2) of tibiae.

Taken together, these results indicate that increased expression of ERRα in the BO2 cell model reduced the formation of osteolytic lesions in bone metastases.

### Example 5

### Regulation of Osteoclast and Osteoblast Formation by ERRα in BO2

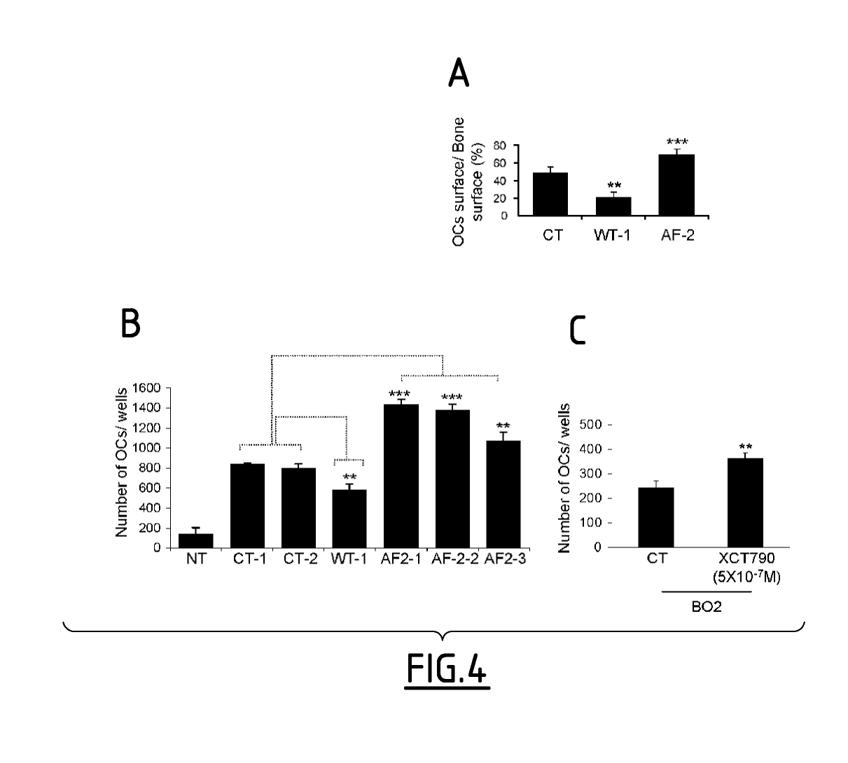

Given the effect of changes in ERRα function in BO2 cells on formation of bone metastases, the inventors next asked whether modulation of ERRα in breast cancer cells alters osteoclasts (OCs), the bone resorbing cells. Tartrate-resistant acid phosphatase (TRAP) staining on tibia sections of BALB/c nude female mice injected with BO2 cell clones showed a decrease in OCs (TRAP-positive) surface per trabecular bone surface (Oc.S/BS) at the bone/tumor cell interface in bone metastases induced by WT-1 cells and an increase with AF2 cells compared to CT cells (FIG. 4A; Table 2). To confirm that modulation of ERRα in breast cancer cells impacts osteoclastogenesis, the inventors treated primary mouse bone marrow cell cultures, which contain OCs precursors, with RANKL and macrophage colony-stimulating factors (M-CSF) together with medium conditioned by CT1 and CT-2, WT-1 or AF2 (1, 2 and 3). Consistent with the in vivo data, the number of TRAP-positive mature multinucleated OCs was decreased in WT-1 cells and increased in AF2 (1, 2 and 3) cells compared to controls (CT1 and CT-2) (FIG. 4B). Similarly to AF2 (1, 2 and 3) results, conditioned media obtained from parental BO2 cells treated with the inverse-agonist XCT-790 which blocks ERRα activity increased OCs number compared to control (DMSO) (FIG. 4C).

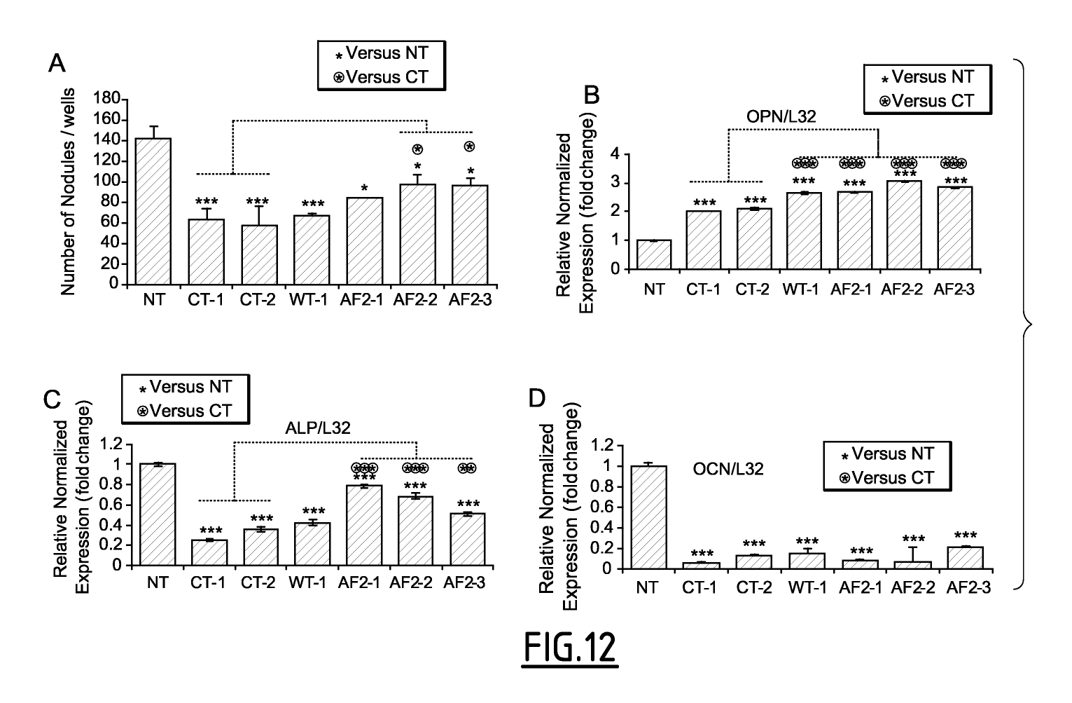

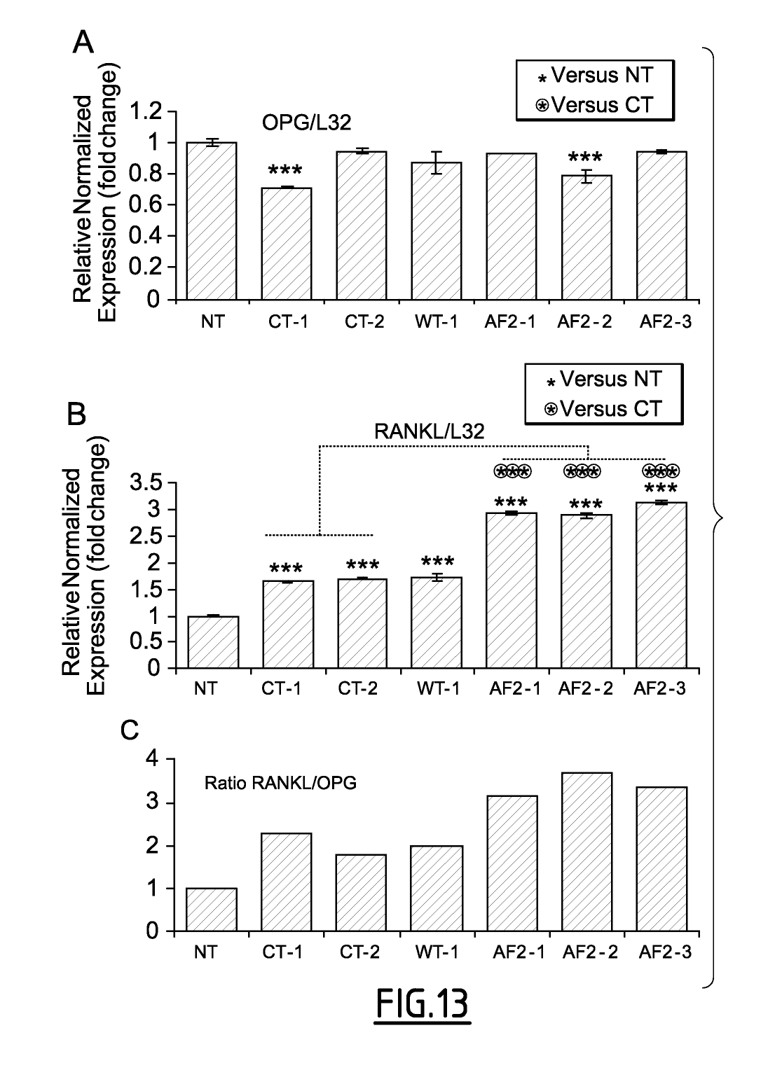

The inventors next asked whether modulation of ERRα activity in BO2 cells alters the number of osteoblasts (OBs), the bone forming cells. Primary mouse calvaria OBs cultured with conditioned media as used above for OCs cells formed fewer mineralized bone nodules (FIG. 12A) irrespective of the conditioning cell type (FIG. 12A; compared non-treated cells (NT)). Consistent with this, expression of alkaline phosphatase (ALP) and osteocalcin (OCN), a mature osteoblast marker expression, were dramatically decreased (FIG. 12C, D). Expression of OPN, an earlier osteoblast marker, was up-regulated in all conditions (FIG. 12B; compared non-treated cells (NT)). On the other hand, reduction in ERRα activity may increased the RANKL/OPG ratio (FIG. 13).

### Example 6

### Regulation of Osteoblast Marker Expression by ERRα in BO2 Cells

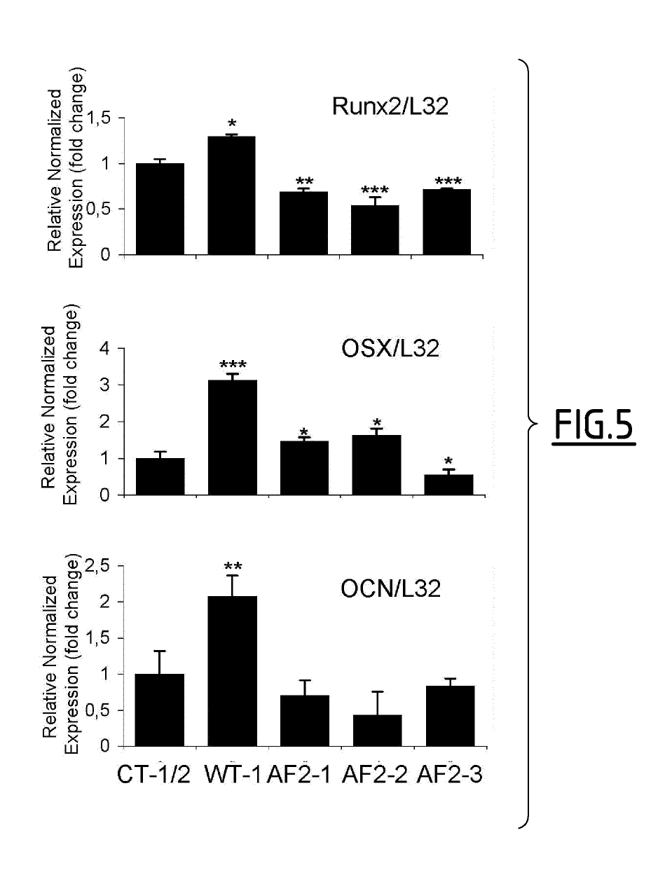

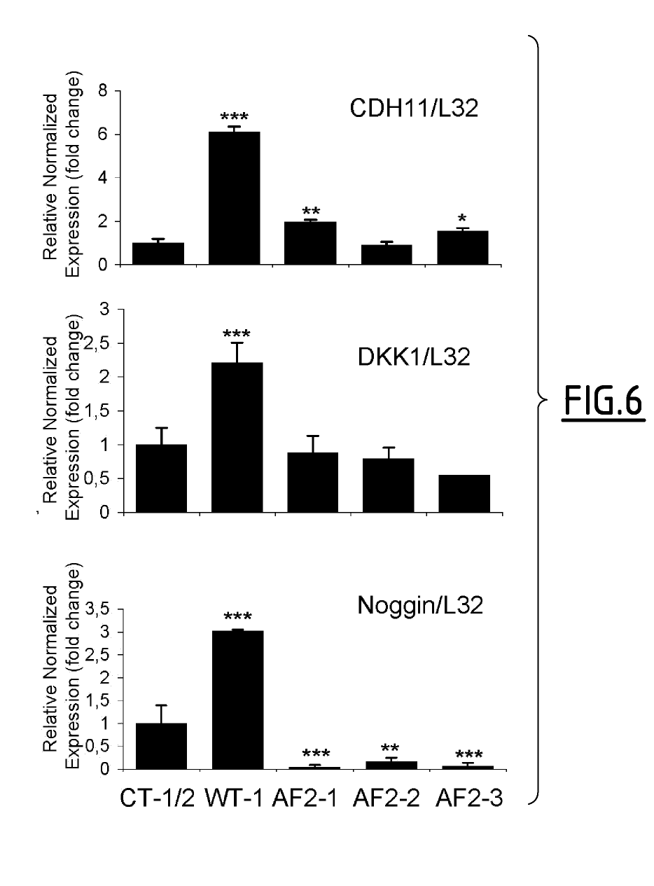

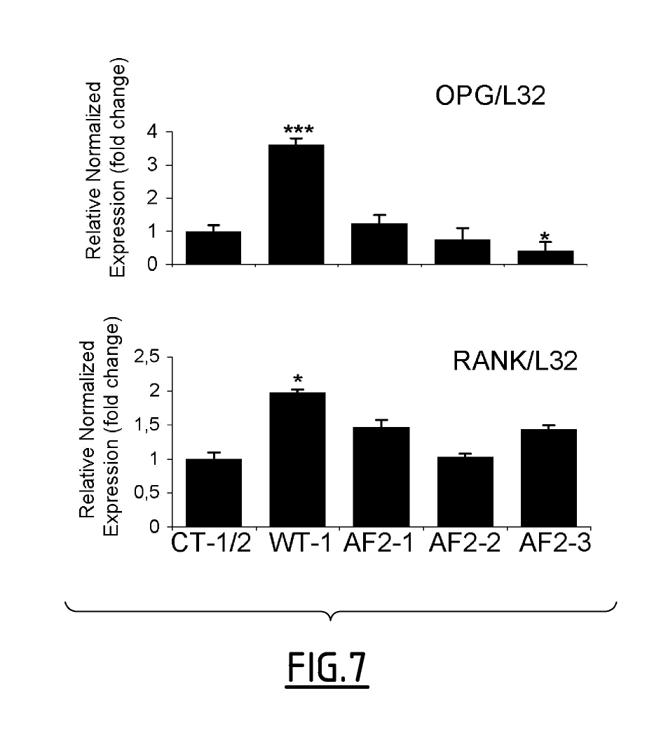

ERRα is involved in OBs differentiation and modulates expression of osteoblast-associated genes such as OPN, bone sialoprotein (BSP), and OCN. Moreover, expression of OBs marker genes by breast cancer cells is now well-established and is hypothesized to be involved in breast cancer cell metastasis to bone. To determine whether modulation of ERRα levels in breast cancer cells alters their capacity to express OBs markers other than OPN (FIG. 3C), the inventors quantified and found that two transcription factors, Runx2 and Osterix (OSX), both master genes in OBs differentiation, are up-regulated in WT-1 cells compared to CT-1/2 cells (FIG. 5-7). Changes in OSX levels in AF2 cells were less consistent, i.e., it was slightly up- or down-regulated in different AF2 clones compared to CT-1/2 cells. Expression of one of the Runx2/OSX target genes, OCN, was up-regulated in WT-1 with a trend towards down-regulation in AF2 (1, 2 and 3) compared to CT-1/2 cells (FIG. 5-7). Similarly, cadherin 11 (CDH11; also known as osteoblast cadherin) was regulated in BO2 cells with different levels of ERRα(FIG. 5-7), suggesting that ERRα may commit BO2 cells into a more osteoblastic phenotype. On the other hand, DKK1 (WNT antagonist) and Noggin (BMP antagonist), both OBs inhibitory factors, were up-regulated in WT-1 and down- or not regulated in AF2 (1, 2 and 3) cells compared to CT-1/2 cells (FIG. 5-7). Paralleling DKK-1 and Noggin expression, the receptor (RANK) of the stimulator of OCs, RANKL, to a small extent and inhibitor OPG to a much greater extent were also regulated by ERRα in BO2 cells (FIG. 5-7). Taken together, the data suggest that ERRα regulates expression of a variety of OBs markers in the BO2 breast cancer model, including factors regulating OBs and OCs formation and overall bone remodeling in vivo.

### Example 7

### OPG and ERRα Association in Breast Cancer Cells

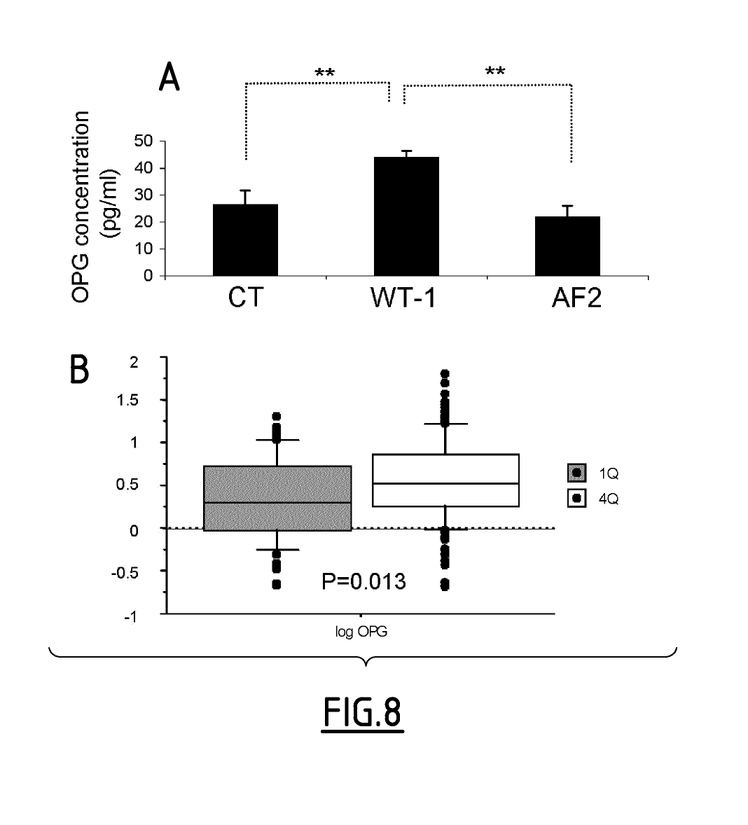

Given the marked alterations that changes in ERRα in BO2 cells elicit in OPG mRNA levels and on OCs in vivo and in vitro, the inventors performed immunohistochemistry on tibia sections of BALB/c nude female mice injected with CT (pool of CT-1 and 2 clones), WT-1 or AF2 (pool of AF2-1, 2 and 3 clones) cells. OPG expression was higher in WT-1 induced metastases than in AF2 or CT cells. OPG secretion as quantified by ELISA was higher in WT-1 compared to CT-1/2 and AF2 cells (pool of AF2-1, 2 and 3 clones) (FIG. 8A).

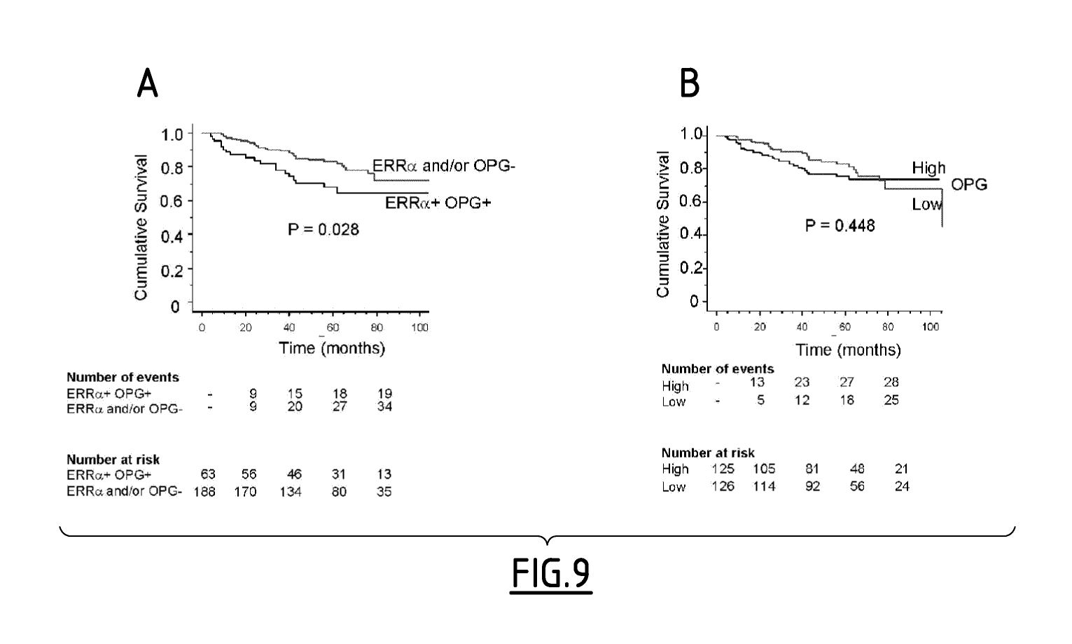

OPG mRNA expression quantified by real time RT-PCR in the cohort of 251 patients (FIG. 1; Table 3) showed a significant association in all patients (N=251) between high ERRαexpression and the median value of OPG (FIG. 8B; Mann-Whitney test, p=0.013). Moreover, there was a significant correlation between high ERRα and OPG (ERRα+/OPG+) mRNA expression and a decrease in relapse free survival (N=251) (p=0.028, log-rank test) (FIG. 9A). Overall survival curves showed no significant correlation between median value of OPG alone and relapse free survival (N=251) (FIG. 9B).

### Example 8

### ERRα Stimulates Tumor Progression and Angiogenesis In Vivo

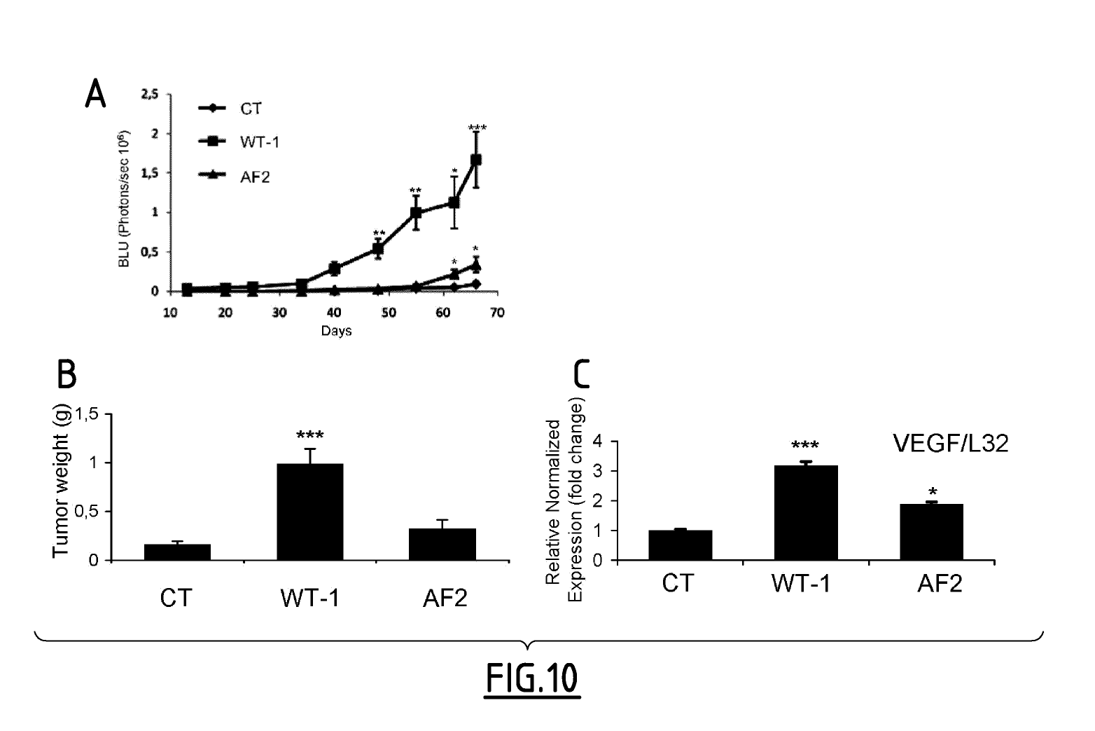

The correlation between high levels of ERRα/OPG and survival suggests a strong impact of ERRα on primary tumor cell expansion. To address this hypothesis, fat pad tumors were induced with CT (pool of CT-1 and 2 clones), WT-1 or AF2 (pool of AF2-1, 2 and 3 clones) cells in NMRI nude female mice. Bioluminescence analysis from day 5-66 revealed dramatically greater tumor progression evident at day 40 in WT-1 cells compared CT or AF2 cells (FIG. 10A). No significant difference was observed between CT and AF2 cells except at day 62 and 66, where a slightly increased tumor burden was observed in AF2 compared to CT cells (FIG. 10A). Tumor weight/size at endpoint (day 66) (Table 2) correlated with the bioluminescence quantification (FIG. 10A), suggesting a pro-tumorigenic function of ERRα in BO2 cells. Interestingly, WT-1 tumors also appeared very highly vascular compared to those derived from CT or AF2 cells, an observation correlating with the levels of VEGF mRNA expression in WT-1- versus AF2- or CT-derived tumors. These data correlate with the significant association observed between high levels of ERRα and VEGF (N=185; p=0.002) in patients (Table 1), reinforcing the view that ERRα is a pro-angiogenic regulator.

### Example 9

### ERRα is Involved in BO2 Cell Invasion

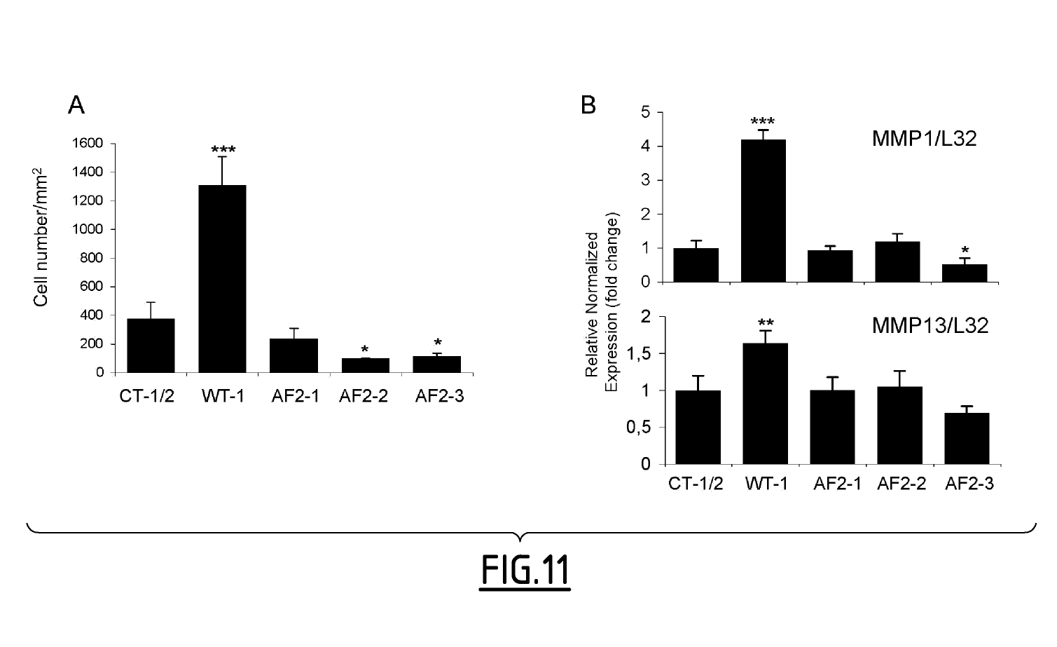

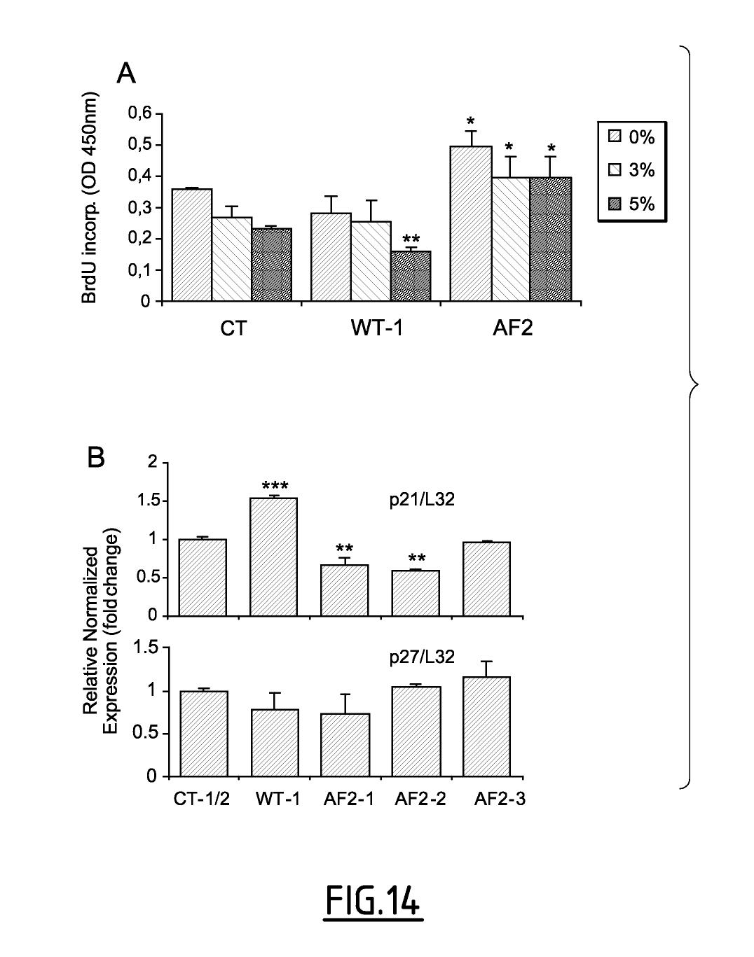

Because ERRα had been implicated in migration and, to a lesser extent, proliferation of breast cancer cells, the inventors next used the BO2 cell models to ask whether ERRα is involved in either process. WT-1 cells were more invasive and AF2 cells less invasive than CT-1/2 cells (FIG. 11A). Consistent with this, expression levels of the mRNAs for the metalloproteinases MMP1 and MMP13 were increased in WT-1 cells and decreased or not different in AF2 cells compared to CT-1/2 cells (FIG. 11B). BrdU incorporation revealed that WT-1 cells were slightly less proliferative, while AF2 cells (pool of AF2-1, 2 and 3) were more proliferative, than CT-1/2 cells (FIG. 14A). The cyclin-dependent kinase inhibitor p21^{WAF1/CIP1} mRNA expression was increased in WT-1 and decreased or not changed in AF2 (1, 2 and 3) cells compared to CT-1/2 cells (FIG. 14B). Neither p27^{KIP1} expression (FIG. 14B) nor apoptosis was affected by ERRα expression levels in the BO2 cell models.

### Example 10

### Discussion

The inventors report here the first evidence that ERRα is involved in development of bone metastases. ERRα over-expression decreased breast cancer cell-induced osteolytic lesion size, inhibited OCs formation and altered expression of a variety of OBs markers, including the main OCs inhibitor OPG (also know to be a pro-angiogenic factor) in breast cancer cells. On the other hand, the inventors also found an association between ERRα/VEGF and ERRα/OPG in a cohort of breast cancer patients (N=251), and the increased size and vascularity of primary tumors in mice inoculated with ERRα-overexpressing breast cancer cells, confirming that ERRα is a pro-angiogenic factor and an unfavorable biomarker in primary breast cancer.

Several previous studies have implicated ERRα in breast cancer. ERRαimmunoreactivity was detected in invasive ductal carcinoma cells and was significantly associated with an increased risk of recurrence, with highest ERRα expression in cancer cells lacking functional ERα. Similarly to previous studies, the inventors found no association between high ERRα expression and menopausal status, tumor size or histological grade in the total patient cohort. The inventors also confirmed both that high ERRα expression correlates with bad prognostic in human breast carcinoma and that high ERRα expression occurs in ER negative tumors. Probably reflecting the high sample number, the inventors also found a statistical association between ERRα expression and histological type and node status in the breast cancer cohort, which was not observed in the studies of (Suzuki et al, 2004, Estrogen-related receptor alpha in human breast carcinoma as a potent prognostic factor, Cancer Res, 64 (13): 4670-6). It is also worth noting that the inventors found a significant association between high ERRα expression and risk of recurrence in ER+ and in the pN0 subset of samples, suggesting that ERRα may be a very useful early prognostic marker in breast cancer. This is further supported by the fact that the inventors observed in the ERRα high group of patients a higher and identical percent of patients that had developed “only” bone metastases (BM) compared to total metastases (including liver, lung, bone and soft tissues) (TM) compared to those with low ERRα expression, suggesting that ERRα is an overall bad prognostic factor that is not a determinant of metastases location of breast cancer cells.

The data with the BO2 breast cancer cell model indicate that high ERRα expression is associated with decreased osteolytic lesions, while inactivated (via the dominant negative ΔAF2) levels of bone destruction dramatically increased. Taken together, the in vivo and in vitro analyses suggest that this reflects mainly the capacity of ERRα to regulate OCs formation (confirmation via the inverse agonist XCT-790). The significant correlation between high ERRα and OPG in patients and the regulation of OPG by ERRα in BO2 cells makes OPG a good candidate for mediating the decrease in osteoclastogenesis in vitro and size of osteolytic lesions in vivo, although the inventors cannot exclude a role for other factors as well.

OPG alone was not associated with relapse free survival or formation of bone metastases in the patient cohort. However, consistent with the increased primary tumor progression in WT-1 cells in vivo, high levels of expression of mRNA for ERRα and OPG (ERRα+/OPG+) correlated with a decrease in relapse free survival. These seemingly paradoxical functions of OPG in breast cancer the inventors attribute to its pro-angiogenic activity in primary tumors and OCs inhibitory activity in bone metastases. Indeed, OPG is a well-known pro-angiogenic factor that increases endothelial cell survival, proliferation, migration and induction of endothelial cell tube formation. Once invasion has occurred, including into bone, OPG is a well-established inhibitor of OCs formation and concomitant decrease in bone degradation and, thereby, expansion of bone metastases. Thus, overexpression of ERRα could be both a bad prognostic marker for primary breast cancer progression but an advantage when breast cancer cells seed into bone.

Simultaneous stimulation of OPG and VEGF, a known ERRα target gene that the inventors also found regulated in the BO2 breast cancer cell model, may explain the highly vascular tumors obtained when WT-1 cells were injected into fat pads or subcutaneously. Interestingly, the data also support recent studies showing that modulating angiogenesis has low impact on bone metastases. Indeed, in the latter studies, hypoxia was reported to be nonessential for bone metastases while promoting angiogenesis in lung metastases and primary tumor growth. Thus, VEGF and the pro-angiogenic role of OPG may have no impact on angiogenesis in bone, as bone is already extremely vascular, but have dramatic impact on vascularization and progression of primary breast tumors or metastases to non-bone sites, providing novel insights into how ERRα can be a bad prognostic factor in the primary tumor (angiogenesis via OPG and VEGF) but a favorable biomarker in the very special case of bone metastases (inhibition of OCs formation through OPG) but not other metastases.

The data also support the view that ERRα in the BO2 cell model regulates OBs differentiation in vitro even though factors secreted by parental BO2 cells inhibit OBs differentiation by blocking lineage progression at an immature stage. Indeed, conditioned medium from the AF2 cell clones increased ALP and a small increase in bone nodule formation concomitant with increased RANKL expression; the associated increase in the RANKL/OPG ratio could contribute to increased bone destruction in vivo. More importantly, ERRα up-regulated expression of various OBs markers in BO2 cells, including Runx2, OSX, OCN and CDH11, suggesting that ERRα commits BO2 cells into a more OBs phenotype and increases BO2 osteomimicry. Concomitantly, ERRαoverexpression up-regulated the OBs inhibitors, DKK1 and Noggin, and the OCs inhibitor OPG, suggesting that overexpression of ERRα can decrease bone remodeling, thus contributing to reduced bone destruction.

How ERRα contributes to cell migration and invasion is also of interest. Overexpression of ERRα increased BO2 cell invasion and correlated with changes in expression of MMP1 and to a lesser extent MMP13, as well as OPN, a known target gene of ERRα that is involved in adhesion and migration. The data are consistent with results of siRNA-mediated knockdown of ERRα in the breast carcinoma MDA-231 cell line which resulted in a dramatic decrease in cell migration. Overexpression of ERRα resulted in only a small change in proliferation suggesting that proliferation is not the main function of ERRα during tumorigenesis in the BO2 model, similar to what was observed in the MDA-231 cells with siRNA knockdown of ERRα. However, the inhibition of WT-1 proliferation in vitro, albeit slight, was associated with stimulation of the cell cycle inhibitor p21 while p27 was not affected. This is in agreement with previous data describing p21 as a target gene of ERRα in promoter-reporter assays in Hela cells, although a recent report has failed to confirm the observation in MDA-MB-231. Differences in co-regulators such as co-activators or post-translational modifications may differentially modulate ERRα activity in different tissues or cells type, a possibility that deserves more attention and that may explain these discrepancies.

In conclusion, the results are consistent with the hypothesis that ERRα is an unfavorable biomarker in breast cancer including at a very early stage of the disease when patients belong to groups of good prognostic (ER+ and pN0), most likely primarily by its regulation of invasion and angiogenesis. The regulation of VEGF and OPG by ERRα in the BO2 cell model supports the view that ERRα is a pro-angiogenic and bad prognostic factor in primary breast tumors and their metastases, with the exception of bone metastases. Indeed, the low impact of VEGF and OPG on angiogenesis in bone together with the ability of ERRα to up-regulate OPG and decrease osteoclastogenesis and overall bone remodeling, support a protective and favourable role for ERRα in osteolytic lesions and bone metastases development.

1. An in vitro method for determining cancer prognosis for a patient suffering from early-stage or low-grade cancer, said method comprising:
a) providing or obtaining a biological sample comprising cancer cells from said patient;
b) measuring the expression level of Estrogen-Related Receptor α (ERRα) in said biological sample comprising cancer cells; and
c) optionally deducing from the result of step b) the prognosis of said patient.

2. The method of claim 1, wherein the patient suffers from hormone-dependent breast cancer and/or is a Estrogen Receptor-positive (ER+) breast cancer patient.

3. The method of claim 1, wherein the patient is a pN0 patient.

4. The method of any of claim 1, wherein the patient is a pN<3 lymph-node positive patient.

5. The method of claim 1, wherein an expression level of ERRα higher than a predetermined threshold is indicative of a poor prognosis.

6. The method of claim 5, wherein an expression level of ERRα higher than a predetermined threshold indicates that the patient is likely to present a short life-expectancy.

7. The method of claim 5, wherein an expression level of ERRα higher than a predetermined threshold indicates that the patient is likely to develop metastases.

8. The method of claim 5, wherein an expression level of ERRα higher than a predetermined threshold indicates that the patient is likely to relapse.

9. An in vitro method for selecting a patient suffering from early-stage or low-grade cancer, and/or from cancer-derived metastasis that is not a bone metastasis, suitable to be treated with a preventive therapy comprising:
a) providing or obtaining a biological sample comprising cells from said cancer or from said metastasis;
b) measuring the expression level of ERRα in said biological sample; and
c) selecting the patient having an expression level of ERRα higher than a predetermined threshold.

10. An in vitro method for determining bone metastases prognosis for a patient suffering from cancer-bone metastases, said method comprising:
a) providing or obtaining a biological sample comprising cells from the primary tumor of said patient;
b) measuring the expression level of ERRα in said biological sample; and
c) optionally deducing from the result of step b) the prognosis of said patient.

11. The method of claim 10, wherein an expression level of ERRα lower than a predetermined threshold is indicative of a poor prognosis.

12. An in vitro method for selecting a patient suffering from cancer-derived bone metastases suitable to be treated with an aggressive therapy comprising:
a) providing or obtaining a biological sample comprising cancer cells from the primary tumor of said patient;
b) measuring the expression level of ERRα in said biological sample comprising cancer cells from said patient; and
c) selecting the patient having an expression level of ERRα lower than a predetermined threshold.

13. The method of claim 12, said method comprising the steps of
a) measuring the expression level of ERRα in said biological sample;
b) comparing the expression level of ERRα in said biological sample to a predetermined threshold; and
c) designing a treatment regimen comprising an aggressive therapy if the expression level of ERRα in said biological sample is lower than the predetermined threshold.

14. A kit comprising:
a) means for detecting the amount and/or expression level of ERRα; and
b) a control sample indicative of the amount and/or expression level of ERRα in a patient suffering from cancer; and
c) optionally, a control sample indicative of the amount and/or expression level of ERRα in a healthy individual.

15. The method according to claim 1, wherein said cancer is breast cancer.

16. The method of claim 9, wherein said cancer-derived metastasis is breast cancer-derived metastasis.

17. The method of claim 10, wherein said cancer-bone metastases is breast cancer-bone metastases.

18. The method of claim 10, wherein said cancer-derived bone metastases is breast cancer-derived bone metastases.

19. The kit of claim 14, wherein said cancer is breast cancer.
